(19) United States
(12) Patent Application Publication
(10) Pub. No.: US 2024/0197177 A1
(43) Pub. Date: Jun. 20, 2024

(54) CONTEXTUALIZED SENSOR SYSTEMS AND METHODS OF USE

(71) Applicant: Aptima, Inc., Woburn, MA (US)

(72) Inventors: John Feeney, Beavercreek, OH (US); Kevin Durkee, Carmel, IN (US); Zachary Kiehl, Tipp City, OH (US); William Depriest, Centerville, OH (US); Matthew Ewer, Dayton, OH (US); Jeremy Ward, Springboro, OH (US); Korey Arvin, Springfield, OH (US)

(73) Assignee: Aptima, Inc., Woburn, MA (US)

(21) Appl. No.: 18/416,761

(22) Filed: Jan. 18, 2024

Related U.S. Application Data
(63) Continuation-in-part of application No. 16/728,972, filed on Dec. 27, 2019.
(60) Provisional application No. 62/785,615, filed on Dec. 27, 2018.

(51) Int. Cl. A61B 5/00

(52) U.S. Cl. CPC A61B 5/0002; A61B 5/681; A61B 5/7264; A61B 5/746; A61B 2560/0242; A61B 2560/0443; A61B 2560/0456

(57) ABSTRACT

A contextualized sensor system (CSS) is provided comprising one or more sensors, one or more memory elements, an alerting library stored in the one or more memory elements, one or more processors, and the one or more memory elements including instructions that, when executed, cause the one or more processors to perform operations comprising: receiving from one of the one or more sensors one or more sensor data, comparing the first sensor data to the alerting library to determine whether an alert situation has occurred, and communicating an alert if the alert situation has occurred. In some embodiments, the CSS further comprises removable sensor modules and component blocks to provide multiple configurations of CSS components. In some embodiments, the component blocks comprise sensors or power sources.

### CROSS-REFERENCE TO RELATED APPLICATIONS

This application is a continuation in part of U.S. patent application Ser. No. 16/728,972, filed on Dec. 27, 2019; U.S. patent application Ser. No. 16/728,972 claims benefit of U.S. Pat. App. No. 62/785,615, filed on Dec. 27, 2018; and all the contents are herein incorporated by reference.

### STATEMENT REGARDING FEDERALLY SPONSORED RESEARCH OR DEVELOPMENT

This invention was made with Government support under Contract Nos. FA8501-16-C-0020, FA8501-16-C-0027 and FA864-92-P-0664 all awarded by the U.S. Air Force. The Government has certain rights in the invention.

### REFERENCE TO SEQUENCE LISTING, A TABLE, OR A COMPUTER PROGRAM LISTING COMPACT DISC APPENDIX

Not Applicable.

### BACKGROUND OF THE INVENTION

### 1. Field of the Invention

This invention relates to sensor systems and methods of use. In some embodiments, the sensor systems are configured to be modular sensors to accommodate multiple sensor configurations and contextualize sensor data related to human physiology and the surrounding environment.

### 2. Description of the Prior Art

The cost, availability, and performance of wearable sensors to monitor physiological and user activities has resulted in an enormous number of commercial off-the-shelf (COTS) wearable sensors. While commonly available, these sensors often have limitations to their effectiveness due to several factors including but not limited to a fixed battery, a fixed set of sensors, a fix communication standard available for transmitting data, and a single location where the sensor can be worn on the body. Additionally, the batteries are not designed to be replaced while the sensor is being worn.

Commonly these wearable sensors are not designed to be configurable in that these limitations of the fixed components make it impossible for user to alter their configuration to accommodate the needs of the user. For example, a heartrate monitor is designed to worn on body with leads attached to the user's chest, powered by a single coin cell battery, and transmits data via Bluetooth.

A need exists to address the shortcomings currently in this field.

### BRIEF SUMMARY OF THE INVENTION

The following summary is included only to introduce some concepts discussed in the Detailed Description below. This summary is not comprehensive and is not intended to delineate the scope of protectable subject matter, which is set forth by the claims presented at the end.

In one example embodiment, a sensor system is provided comprising a mounting base, a body area subsystem comprising a sensor module, the mounting base configured to removably couple the sensor module to the mounting base, and the sensor module comprising: a sensor subsystem, a communication subsystem configured to transmit raw data captured from the sensor subsystem and receive contextualized data, and a power subsystem configured to power the sensor subsystem and the communication subsystem.

In some embodiments, the sensor module houses the power subsystem, the sensor subsystem and the communication subsystem, and the sensor module is configured to removably couple the packaged to the mounting base whereby a first sensor module with a first sensor coupled to the mounting base can be exchanged with a second sensor module with a second sensor.

In some embodiments, the sensor module houses the power subsystem, the sensor subsystem and the communication subsystem, the sensor module is configured to removably couple the sensor module to the mounting base whereby a first sensor module with a first sensor coupled to the mounting base can be exchanged with a second sensor module with a second sensor, and the mounting base is configured to be mounted on a limb of a worker whereby the first sensor module can be exchanged with the second sensor module while the mounting base is mounted on the limb of the worker.

In some embodiments, the sensor subsystem comprises an optical sensor, and the mounting base comprising an opening configured to allow the optical sensor to be proximal to a portion of a skin of a wearer when the optical sensor is coupled to the mounting base and the mounting base is mounted on a limb of a wearer.

In some embodiments, the sensor subsystem comprises a sensor selected from the group consisting of an environmental sensor, a location sensor, a physiological sensor, a behavior sensor, and a posture sensor.

In some embodiments, the mounting base further comprises a base coupling element configured to removably couple the sensor module to the mounting base.

In some embodiments, the sensor module further comprises a sensor module coupling element configured to removably couple the sensor module to a component block, the component block comprising a component block coupling element configured to removably mate with the sensor module coupling element, the component block coupling element comprises one or more pliant clasp, and the sensor module coupling element comprises one or more recesses to receive a portion of the pliant clasp.

In some embodiments, the component block comprises one selected from the group consisting of a sensor component block, a communication component block, a power component block, a battery clock component block, and an antenna component block.

In some embodiments, the component block comprises a sensor component block comprising a sensor selected from the group consisting of an environmental sensor, a location sensor, a physiological sensor, a behavior sensor, and a posture sensor.

In some embodiments, the component block comprises a communication component block comprising a communication protocol module programmed to communicate according to a communication protocol selected from the group consisting of a Bluetooth protocol, a 802.11 protocol, and a LoRa protocol.

In some embodiments, the sensor system further comprises a monitoring subsystem configured to determine a contextualized state of a worker from a sensor data from the sensor module.

In some embodiments, the sensor system further comprises a monitoring subsystem comprising a situation classifier configured to determine a contextualized state of a worker from a sensor data from the sensor module.

In some embodiments the modularity of system components with the addition of the swapable optical sensing component and/or an additional component blocks, the CSS can be configured to provide multiple sources of data from a single device and single body location where it is being worn.

In some embodiments, the sensor system further comprises a monitoring subsystem in communication with the communication subsystem; the monitoring subsystem configured to receive a first sensor data from a first sensor of the sensor module for a time and a second sensor data from a second sensor of the sensor module for the time; the monitoring subsystem comprising: an expert subsystem comprising a situation classifier module and an intervention/alert module, a subsystem database comprising contextualization rules and a library of alert rules, the situation classifier module is configured to determine a first contextualized situation at the time from the first sensor data using the contextualization rules, the situation classifier module is configured to determine a second contextualized situation at the time from the first sensor data and the second sensor data using the contextualization rules, the intervention/alert module configured to compare the first contextualized situation to the library of alert rules to determine whether an alert situation has occurred at the time, and the intervention/alert module configured to compare the second contextualized situation to the library of alert rules to determine whether the alert situation has occurred at the time; whereby if the alert situation has occurred at the time based on the first contextualized situation, but the alert situation has not occurred at the time based on the second contextualized situation, the monitoring subsystem configured to not communicate an alert; and whereby if the alert situation has occurred at the time based on the first contextualized situation, and the alert situation has occurred at the time based on the second contextualized situation, the monitoring subsystem configured to communicate an alert.

In one example embodiment, a processor based contextualized sensor system is provided comprising a body area subsystem comprising: a first sensor configured to communicate a first sensor data from a time, and a second sensor configured to communicate a second sensor data from the time; a monitoring subsystem comprising: a message broker configured to receive the first sensor data, the second sensor data and the time from the body area subsystem, an expert subsystem comprising a situation classifier module and an intervention/alert module, a subsystem database comprising contextualization rules and a library of alert rules, the situation classifier module is configured to determine a first contextualized situation at the time from the first sensor data using the contextualization rules, the situation classifier module is configured to determine a second contextualized situation at the time from the first sensor data and the second sensor data using the contextualization rules, the intervention/alert module configured to compare the first contextualized situation to the library of alert rules to determine whether an alert situation has occurred at the time, and the intervention/alert module configured to compare the second contextualized situation to the library of alert rules to determine whether the alert situation has occurred at the time; whereby if the alert situation has occurred at the time based on the first contextualized situation, but the alert situation has not occurred at the time based on the second contextualized situation, the monitoring subsystem configured to not communicate an alert; and whereby if the alert situation has occurred at the time based on the first contextualized situation, and the alert situation has occurred at the time based on the second contextualized situation, the monitoring subsystem configured to communicate an alert. In some embodiments, the first sensor comprises a physiological sensor configured to communicate a physiological data as the first sensor data, the situation classifier module further comprises a state contextualizer module configured to determine a first contextualized state of a worker at the time from the first sensor data and a second contextualized state of the worker at the time from the first sensor data and the second sensor data, the first contextualized situation comprises the first contextualized state of the worker at the time, and the second contextualized situation comprises the second contextualized state of the worker at the time. In some embodiments, the body area subsystem further comprises a mounting base, and the first sensor and the second sensor are configured to be removably coupled to the mounting base.

In one example embodiment, a processor based contextualized sensor system is provided for determining a contextualized state of a worker, the system comprising a body area subsystem comprising: a mounting base, a sensor module comprising one or more sensors configured to collect a state data of a worker, the state data comprising a first raw state data at a time and a second raw state data at the time, and a communication subsystem configured to communicate the first raw state data, the second raw state data and the time; a monitoring subsystem comprising: a message broker configured to receive the first raw state data, the second raw state data and the time from the body area subsystem, an expert subsystem comprising a situation classifier module, a subsystem database comprising contextualization rules, and the situation classifier module is configured to determine a raw state of the worker at the time from the first raw state data and determine a contextualized state of the worker at the time from the first raw state data at the time and the second raw state data at the time and the contextualization rules; the contextualization rules comprise a raw variable and a contextualization variable to identify a resulting contextualized variable; the raw variable comprises the first raw state data of the worker at the time; the contextualization variable comprises the second raw state data of the worker at the time; the resulting contextualized variable comprises an objective representation of the contextualized state of the worker at the time given the first raw state data at the time and the second raw state data of the worker at the time; and the situation classifier module is configured to use the contextualization rules to (1) confirm the raw state of the worker at the time as the contextualized state of the worker at the time or (2) override the raw state of the worker at the time as the contextualized state of the worker at the time. In some embodiments, the sensor module comprises a first sensor and a second sensor, and the first sensor and the second sensor are configured to be removably coupled to the mounting base.

In one example embodiment, a contextualized sensor system (CSS) is provided comprising one or more sensors, one or more memory elements, an alerting library stored in the one or more memory elements, one or more processors and the one or more memory elements including instructions that, when executed, cause the one or more processors to perform operations comprising: receiving from one of the one or more sensors one or more sensor data, comparing the first sensor data to the alerting library to determine whether an alert situation has occurred, and if the alert situation has occurred, communicating an alert. In some embodiments, the one or more sensors comprises an environmental sensor, a location sensor, a physiological sensor, a behavior sensor and a posture sensor, and the one or more sensor data comprises an environmental data, a location data, a physiological data, a behavior data and a posture data. In some embodiments, the operations further comprise contextualizing one or more of the environmental data, the location data, the physiological data, the behavior data and the posture data.

### BRIEF DESCRIPTION OF THE SEVERAL VIEWS OF THE DRAWINGS

In order that the manner in which the above-recited and other advantages and features of the invention are obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1A shows a functional diagram illustrating an architecture of one example embodiment of a Contextualized Sensor System (CSS);

FIG. 1B shows a system diagram illustrating an architecture of one example embodiment of a contextualized sensor system (CSS);

FIG. 2A shows a system diagram illustrating an architecture of one example embodiment of a body area subsystem;

FIG. 2B shows a functional diagram illustrating an architecture of one example embodiment of a body area system;

FIG. 3A shows a functional diagram illustrating an architecture of one example embodiment of a sensor subsystem;

FIG. 3B shows a functional diagram illustrating an architecture of one example embodiment of a power subsystem;

FIG. 3C shows a functional diagram illustrating an architecture of one example embodiment of an executive subsystem;

FIG. 3D shows a functional diagram illustrating an architecture of one example embodiment of a user interface;

FIG. 3E shows a functional diagram illustrating an architecture of one example embodiment of a communication subsystem;

FIG. 4A shows a functional diagram illustrating an architecture of one example embodiment of a sensor socket subsystem;

FIG. 4B shows a functional diagram illustrating an architecture of one example embodiment of a communication socket subsystem;

FIG. 5A shows a top view of one example embodiment of a mounting base;

FIG. 5B shows a top perspective view of one example embodiment of a mounting base;

FIG. 6 shows a top perspective exploded view of an example embodiment of a mounting base, a sensor module and a component block;

FIG. 7A shows an exploded view of one example embodiment of a sensor module and a component block;

FIG. 7B shows a view of a component block switching with a sensor module;

FIG. 8A shows a view of a sensor subsystem with an optical component block inserted into the sensor module;

FIG. 8B shows a view of an optical component block switching into the sensor module;

FIG. 9A shows a view of how a charging cable with a POGO pin connection would connect to a component block;

FIG. 9B shows a view of how a charging cable with a POGO pin connection would connect to the sensor module; and

FIG. 10 illustrates one example embodiment of a computer system suitable for a contextualized sensor system (CSS).

### DETAILED DESCRIPTION OF THE INVENTION

COPYRIGHT NOTICE: A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. The following notice applies to any software and data as described below and in the drawings hereto: Copyright® Aptima, Inc., 2020-2024, All Rights Reserved.

Contextualized sensor systems (CSS) and methods of use will now be described in detail with reference to the accompanying drawings. It will be appreciated that, the following description focuses on a system that provides a contextualized sensor systems and methods disclosed herein have wide applicability. For example, the CSS described herein may be readily employed in situations where traditional sensor systems are not suitable for use in measuring the users and the environment around them. Notwithstanding the specific example embodiments set forth below, all such variations and modifications that would be envisioned by one of ordinary skill in the art are intended to fall within the scope of this disclosure.

As used herein, the term “subsystem” refers to a distinct functional component within a larger system. The operations performed by a “subsystem” are a specialized area of operation within an overall system architecture contributing to the overall functionality and performance of the entire system. A “subsystem” may be comprised of hardware, middleware, and software components, and does not include operations performed by a human being.

As used herein, the term “module” refers to hardware and/or software implementing entities and does not include a human being. The operations performed by the “module” are operations performed by the respective hardware and/or software implementations, e.g. operations that transform data representative of real things from one state to another state, and these operations do not include mental operations performed by a human being.

As used herein, the term “executive” refers to hardware and/or software implementing entities used to control or configure system components and does not include a human being. The operations performed by the “executive” are operations performed by the respective hardware and/or software implementations, e.g., configuration commands to a subsystem to transmit data with one or more protocols and these operations do not include mental operations performed by a human being.

As used herein, the term “sensor” is a broad term and is to be given its ordinary and customary meaning to a person of ordinary skill in the art (and are not to be limited to a special or customized meaning), and furthermore refers without limitation to any device comprising the capability to detect or measure a physical property, record, indicate, or transmit the measure.

As used herein, the term “sensor data” is a broad term and is to be given its ordinary and customary meaning to a person of ordinary skill in the art (and are not to be limited to a special or customized meaning), and furthermore refers without limitation to any data associated with a sensor, such as but not limited to an electrocardiogram (ECG) monitor, inertial measurement unit (IMU), force sensing resistors (FSR), electromyography (EMG) monitors, thermometer, hygrometer, goniometer, a camera device, a heart rate monitor, an accelerometer, a photodetector and a reflectance-based pulse rate monitor.

As used herein, the term “component block” is a broad term and is to be given its ordinary and customary meaning to a person of ordinary skill in the art (and are not to be limited to a special or customized meaning), and furthermore refers without limitation to any standardized component of a sensor, such as a battery component block, a communication component block, a sensor component block or an antenna component block.

The term confined space, as used herein, is a broad term and is to be given its ordinary and customary meaning to a person of ordinary skill in the art and furthermore refers without limitation to areas that are considered “confined spaces” because while they are not necessarily designed for people, they are large enough for workers to enter and perform certain jobs. A confined space may also have limited or restricted means for entry or exit and is not designed for continuous occupancy. Confined spaces include, but are not limited to, tanks, vessels, silos, storage bins, hoppers, vaults, pits, manholes, tunnels, equipment housings, ductwork, pipelines, etc. A confined space may also have two or more of the following characteristics: contains or has the potential to contain a hazardous atmosphere; contains material that has the potential to engulf an entrant; has walls that converge inward or floors that slope downward and taper into a smaller area which could trap or asphyxiate an entrant; or contains any other recognized safety or health hazard, such as unguarded machinery, exposed live wires, or heat stress.

### The Technical Problem

One technical problem this solution addresses is how to automatically reconfigure and operate a CSS when either a required sensor or sensors changes or there is a requirement to change the means by which the sensor system needs to communicate. This challenge presented by this problem is increased when the sensor system must be worn by a user to capture and transmit sensor data from based on the sensors being worn by a user.

For example, in one particular environment, a sensor system with a heart rate monitor transmitting data via Bluetooth works to collect and transmit the user's heart rate. However, there are well defined ranges by which reliable and accurate transmission of data is achievable by Bluetooth. These transmission distances may be overcome when a different communication protocol, such as Long-Range Wireless (LoRa) is used. In this example, when transmission ranges exceed the Bluetooth protocol capability, the user would swap out the Bluetooth communication component with a LoRa communication component. The newly configured contextualized sensor would automatically begin the transmission of the users heart rate over LoRa. Similarly, when a different sensor is required, the user can swap out the current sensor component to reconfigure the CSS to transmit sensor data associated with the newly installed sensor component.

Sensors are becoming ubiquitous across nearly every facet of everyday life—sensors to track how much ink is available in a printer, sensors to control lights, sensors to assist with parking your car. Sensors may be part of a closed loop system where the sensor controls something (e.g., turning on/off lights) without any human intervention. Sensors may also be part of a human-in-the-loop system that provides data that is presented for a human to act upon the data.

Wearable sensors are a class of sensors that are designed and packaged to allow humans or animals to don them to collect sensor data of interest (e.g., heartrate monitors, step counters, smartwatches, etc.). This class of sensors can have more than one sensor packaged together into an integrated unit. While this allows for manufacturers to leverage economy of scale to reduce costs, it means that wearable sensors are locked into specific sensors, battery size/weight/performance, and communication modes (e.g., Bluetooth, Wi-Fi, proprietary wired connections).

Wearable sensors are inflexible in terms of the sensors they provide, the battery performance available, and the mode by which they communicate their sensor data. This limitation results in forcing users to accommodate the limitations of available wearable sensors. Currently wearable sensors are not produced with modular components that allows users to select and modify the wearable sensor to meet their needs in terms of sensing, power, and communication. This results in several technical problems which includes but not limited to: sensors cannot be customized to meet need or to allow unconventional sensing capabilities (e.g., a heartrate monitor with a volatile organic compound sensor); batteries cannot be selected to meet the power requirements of a user (e.g., integration of a larger battery allowing for long duration use); and using a particular communication protocol (e.g., swapping out Bluetooth to LoRa communication to support long range transmission/receiving).

### The Technical Solution

The technical solution to address the above technical problems is generally to create a body area subsystem with a mounting base, a sensor module and component blocks designed to accommodate sensors, power, and communications allowing for a configurable wearable sensor. The components may be worn on a limb of a worker, in their clothing or in close proximity to the worker. This solution addresses needs for modularity and integration of sensor components so that appropriate data can be communicated and analyzed by a monitoring system. The solution has the capacity to create or modify a wearable sensor by interchanging sensor modules or combining with component blocks to meet the needed sensing, power, and communication needs of the user.

Embodiments of the technical solution provide: (1) a means of physically removing and connecting sensor modules to a mounting base, (2) a universal electronics interface between the sensor module, component blocks and other system components, (3) a physical specification for the configuration of the sensor module and component blocks to allow component blocks to house sensor or other system components and to allow them to be removably coupled to the sensor modules, and (4) an interface to an external antenna which would enhance the performance of the wearable sensor's range of communication.

The unique geometric layout of the sensor modules provides a means to securely couple different sensor modules to the mounting base. The layout of the sensor modules also provides coupling elements that securely couple component blocks and allow these component blocks to interact and communicate with other CSS components.

The universal electronics interface for the sensor module and component blocks is addressed through a common layout of three types of electronic connections which includes: (1) power connections to include power (PWR) and ground (GND), (2) data connections to include transmit data (TXD), receive data (RXD), and four addition connections that can be used to accommodate additional data connections; and (3) status connectors to include a connection status (STA) of the component, and enable connector (EN) that can be used to toggle the component powered on/off.

The configuration of the sensor modules and component blocks accounts for current and future electrics or electronic components that can be packaged into these components. The physical specification for this configuration has inherent tradeoffs which includes designing the size and shape such that it has the ability to securely physically couple sensor modules and component blocks and is physically large enough to house and encapsulate the electronics required while being in a form factor where users will be willing to don the wearable sensors for long periods of time.

The connection to an external antenna may improve the communication range and or performance and accounts for non-traditional antenna configurations which could be integrated into a flexible media—such as a watchband, clothing, or worn accessory.

When the CSS includes monitoring systems, multiple sources of data may be used, such as but not limited to: (1) location data, (2) environmental data, (3) physiological data, (4) behavioral data, and (5) physical orientation (e.g., posture) data. While there are intersections between these data sources (by design), each data source may provide insight into the underlying situation or condition.

The sensors serve as initial inputs to the system, as additional processing and fusion is performed before a determination of worker state, posture, or activity can be ascertained.

Generally, the sensors provide raw data that is processed to produce measures of interest for use within the subsequent steps of the process. After the sensor data is collected and processed, measures are computed. These measures are then fused with each other to align them both temporally and by location. The temporal fusion process ensures measures are combined concurrently and in the correct temporal order. Likewise, the fusion of location data ensures that measures and sensors are collocated in the same space. For example, physiological and motion data can be fused with atmospheric data based on the location data of the worker.

Once fused, the measures may then be used in two functionalities. The first function of these measures is to serve as an input to predictive models to provide early-warning alerts. These predictive models focus on the prediction of trends in key data sources and measures, such as cardiovascular data and heart rate. The predictive models are used in conjunction with the real-time data values to prime alerting rules to generate alerts when the predictive models have a high confidence that an alerting situation is approaching. The predicted values and additional rule-based logic are used to generate an alert to indicate the potential for an adverse worker state. The second function of the measures is to assist in real-time monitoring and alerting. This function is achieved by the utilization of a library of clinically derived alerting rules that were developed based on a team of medical clinicians. More specifically, this library focusing on describing specific physiological conditions that could be assessed using the data available.

And by utilizing modular sensors and configurations, the CSS can be configured to provide multiple types of sensor data to assist in the predictive modeling, monitoring and alerting. In some embodiments, the modular sensor configurations can be provided in sensor kit configured to provide sensor data for specific environments and situations.

In summary, embodiments of the described solution use a fusion of disparate data sources to create a more accurate system for health status alerting within confined spaces or environments otherwise not meant for human habitation.

### Differences from Prior Solutions

The disclosed CSS is configured to integrate multiple sources of sensor data (e.g., location, environmental, physiological, behavioral, and orientation/posture data) to provide context to a human health and safety sensing system which is different from many prior art solutions when the specific use case of health and safety monitoring is within a confined space. There are very few health and safety monitoring systems that focus on typical work activities that comprise confined space work. Out of the few systems that do exist, the inventors are unaware of any that use sensor fusion and contextual data as a method to mitigate the number of false positive alerts. Other existing systems may exist incorporate one or two data sources; however, they typically assume normative conditions during their sampling. For example, typical use of a COTS heart rate monitor assumes normal postures and atmospheric conditions. While these assumptions are acceptable for many applications, they are not acceptable within a capricious and potentially dangerous environment, such as that of an aircraft fuel tank.

Another difference from prior art is that the CSS accounts for differences in baseline physiology. There is a fair amount of between-subject variability in baseline physiology and behavior. Additionally, there is often even day-to-day variability for the same individual. This observation is accounted for in the current system design by the collection of baseline data at the beginning of each worker's shift, which is ultimately used to calculate individualized alerting thresholds.

In summary, the fusion of sensor data, contextual information, and worker baseline information allows for the development of a hierarchical alerting paradigm that is intelligent and adaptable. Though some of these features may exist in other systems, the exist in isolation to the best of our knowledge.

### The Practical Application

The CSS recognizes and solves technical problems related to monitoring humans working in unusual situations.

The CSS enables real-time sensing of workers and their surrounding environments as they operate in confined spaces and other potentially hazardous areas. CSS supports prevention, detection, and intervention of health and safety hazards while reducing the time, costs, and manpower required by current confined space monitoring practices. This solution has an objective of providing capabilities to report, view, and control all factory operations and resources across different facilities, and to enhance depot productivity through reduced machine and process downtime. Compared to current-day operations that require safety attendants at a one-to-one ratio, CSS allows a many-to-one ratio of workers per safety attendant, thereby reducing costs and increasing efficiencies. The CSS may provide an enterprise-wide solution that reduces costs, improves performance, and increases reliability across all weapon systems.

For example, the disparate data sources described above are combined to be important indicators of worker state in unconventional postures. The utilization of five sources of data and subject matter expert (SME) recommendations is used to derive alerting rules to produce non-intuitive results. The derived rules are not simple syllogism of individual sensor data, but rather a complex integration of the different data sources, which ultimately results in fewer false alarms and increased specificity. The derived rules account for a complex interplay between the actions of the workers, their expected physiological response to these activities, and the impact of the environmental conditions they find themselves in.

Additionally, the resulting physiological data of workers in unconventional orientation/postures doing unconventional activities can differ from conventional postures and activities, such as sitting or standing. Changes in physical posture can skew certain physiological signals (e.g., digit-derived photoplethysmogram). For example, compression of the diaphragm may result in lower respiration rates or respiratory tidal volume.

Without the integration of data such as posture, position, and activity, the resulting suppression in respiration could result in increased false alarms. Likewise, the combination of unconventional postures and activities could lead to an increased heart rate, thus erroneously indicating a state of distress.

By integrating the data sources and understanding the normal underlying physiological responses of workers in unconventional postures doing physically exerting tasks, it is possible to assess worker state more accurately.

This solution also uniquely incorporates the features of new sensors and communication protocols. Prior to the miniaturization of requisite components and availability of short-range wireless communication protocols, real-time multi-modal data acquisition and transition simply would not have been possible.

The use of components blocks, and their configuration for modularity, provides a number of key features both for the designers and the users of sensors. For the designers, modularity allows for flexibility allowing specific functionality by swapping out sensor components or the additional of component blocks. This flexibility provides a pathway to upgrade specific parts as new technologies become available. Additionally, modularity supports cost-effective development while simplifying testing and troubleshooting of devices since the prior components, resulting in faster time to market.

For users, the modularity allows for customization and personalization to their specific needs and use cases. Additionally, modularity supports upgrading of devices and facilitating easier repairs both of which increases the lifespan of the sensor since it can be updated, repaired, or enhanced.

### One Embodiment of the Contextualized Sensor System

The described contextualized sensor systems for context-enabled multi-sensor fusion for optimized health and safety alerting during unconventional physical postures is comprised of a system with numerous components. At a high level, the system uses an architecture for routing the requisite data along with a context-enabled hierarchical alert paradigm. Within the architecture, there are numerous subcomponents, such as sensors for different data sources, software modules for processing the data, and devices for relaying both data and health status alerts.

Example applications of the CSS include but are not limited to: supporting multiple operationally relevant roles charged with ensuring the health and safety of individuals operating in confined spaces; providing adequate, clear, and role-appropriate situational awareness regarding confined spaces, entrants, and roving attendants in monitored areas; providing detection and actionable alerting of states and events that pose a potential risk to the health and safety of individuals operating in confined spaces; providing a real-time monitoring station software that enables a person to perform the roles and responsibilities of a remote attendant from a remote location; providing capabilities that enable a person to perform the role and responsibilities of a roving attendant on an as needed/floating basis; providing support for emergency responders to be notified and provided with actionable information in response to a person's request for emergency intervention; providing clear and distinct real-time physiological information about individuals operating in confined spaces; providing clear and distinct real-time atmospheric sensing information within the confined spaces just prior to and during the entire time individual(s) are operating within those spaces; providing clear and distinct real-time awareness of which individual(s) are currently in each confined space; providing clear real-time awareness of the locations of roving attendants working in the monitored areas; providing a means for a confined space entrant to communicate their intent and status clearly and simply to the appropriate person(s); being able to detect and alert in real-time potential risks to health and safety using the sensor data available to the system; being able to detect and alert in real-time potential risks to health and safety due to inadequate or faulty sensor data; being able to detect and alert in real-time potential risks to health and safety due to loss of connectivity between system components; and being able to detect and alert in real-time when individual(s) are in confined spaces without access approval or otherwise required to exit the space.

### System Overview

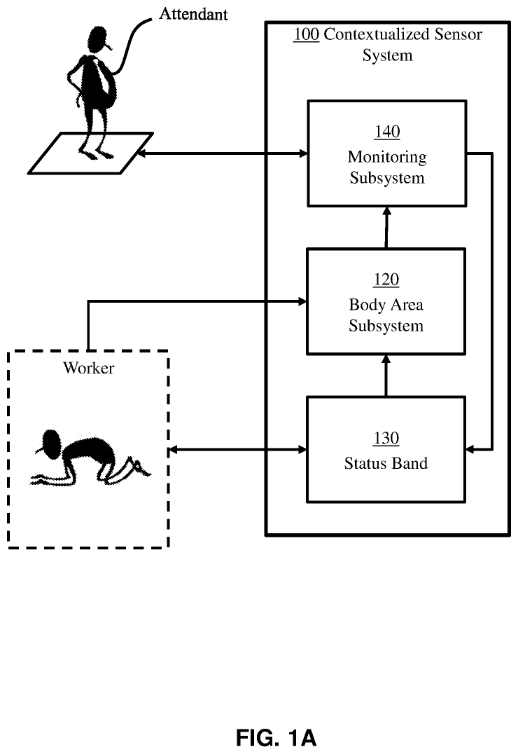

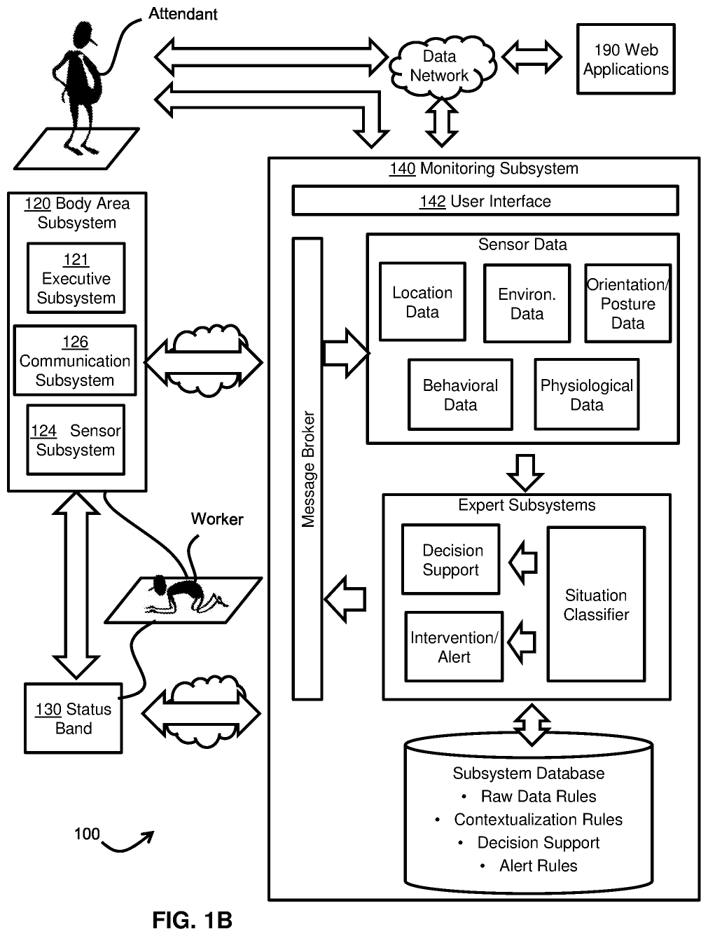

The overall architecture of one example embodiment of the CSS is shown in FIG. 1A. As shown, the CSS 100 comprises a body area subsystem (BAS) 120, a status band 130 and a monitoring subsystem 140. The body area subsystem 120 is generally worn by the worker generates and transmits data sources from the worker and the monitoring subsystem 140 uses this data to classify the state of the worker and provides decision support or interventions based on that state. The status band 130 is typically worn by the worker and provides an interface from the monitoring subsystem 140 to the worker and may include special alerting interfaces. Optionally, the CSS 100 may further comprise web applications that allow the CSS 100 to communicate information to an attendant or other remote users.

A more detailed architecture of one example embodiment of the CSS is shown in FIG. 1B. In this example embodiment, the personal or BAS 120 may comprise a sensor subsystem 124, a communications subsystem 126 (also referred to as a receiver/transmitter) and an executive subsystem 121 (also referred to as BAS applications). The sensor subsystem 124 generally captures data local from the worker, the communication subsystem 126 communicates this data to the monitoring subsystem 140 and the executive subsystem 121 provides configuration and other features to the BAS 120. The communication subsystem 126 also receives data from the monitoring subsystem 140 such as alerts and status data.

In some embodiments, the status band 130 generally provides a local status to the worker. The status band 130 may communicate alerts to the worker and may allow the worker to provide additional data to the monitoring subsystem 140.

In some embodiments, the monitoring subsystem 140, also called the CSS server, generally enables the CSS 100 to analyze sensor data, share information between all other components, save data, and preserve system state. The monitoring subsystem 140 may include components such as: a user interface 142 to allow users to interface with the monitoring subsystem 140, expert subsystems to analyze the sensor data received from the body area subsystem 120, a system database to provide rules and other tools to analyze the sensor data and a message broker to communicate with the body area subsystem 120.

In some embodiments, the web applications 190 may be provided to allow for storage of data and allow for remote status and remote management of the CSS 100, CSS subsystems, algorithms, and data.

Operationally, the activity flow of the CSS generally comprises receiving sensor data from BAS sensor subsystem 124 and transmitting that with the BAS communication subsystem 126 as sensor data to the monitoring subsystem 140 for data interpretation and fusion, determining contextualized states by the expert subsystems. In some embodiments, the sensor data may be pre-processed within the BAS 120 and transmitted to the monitoring subsystem. This pre-processing may include processing such as but not limited to down sampling of raw data to compute an average of raw data, windowing of raw data to smooth the data, time synching of the data, and error detection of the data. The expert subsystems will take the fused data together with alerting data from the subsystem database to determine whether an alarm state exists. If an alarm state exists, an alert will be communicated to the BAS 120 and/or status band 130 and/or attendant user interface 142. In some embodiments, the CSS may communicate system information to web applications 190 for remote monitoring of the data.

### Body Area Subsystem (BAS):

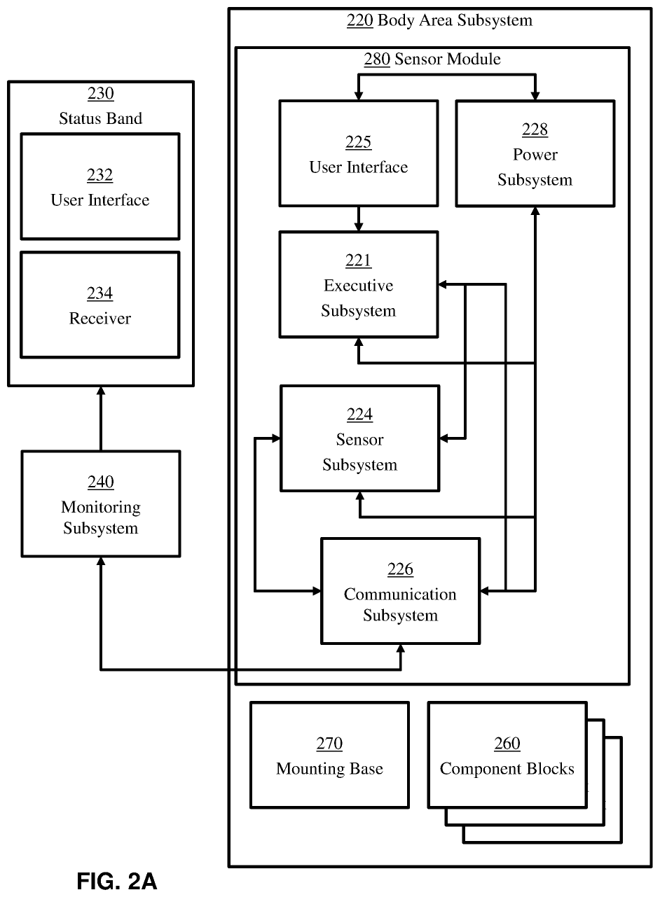

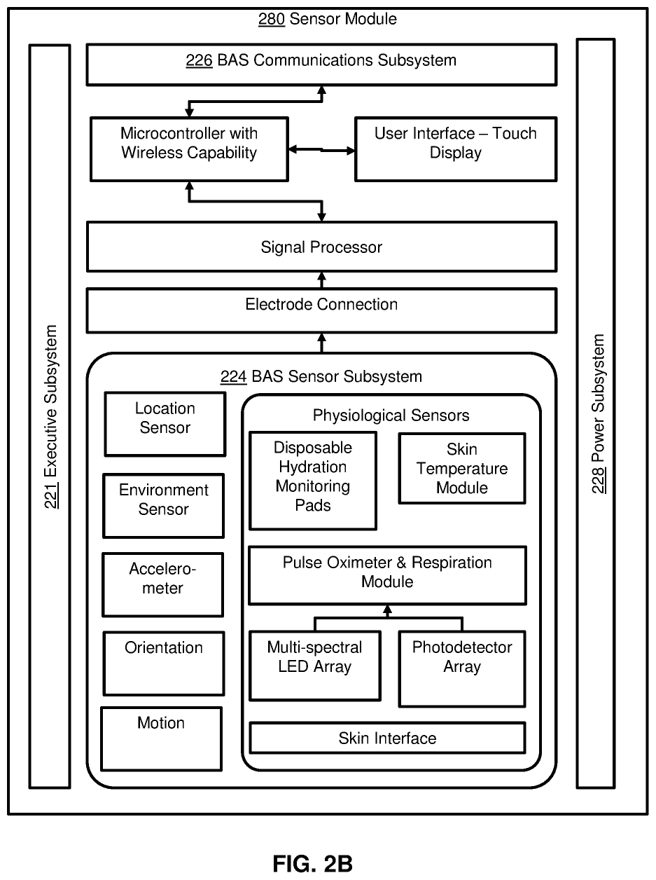

Referring to FIG. 2A, the BAS 220 generally serves as the data source from its sensor subsystem 224 to the monitoring subsystem 240, and to the status band. The BAS 220 may also provide additional contextualization information for the system. The BAS 220 generally comprises the sensor module 280, the mounting base 270 and optionally one or more component blocks 260. The sensor module 280 may be configured as a component block. The sensor module 280 may comprise the sensor subsystem 224, the communication subsystem 226 and the executive subsystem 221, the sensor module user interface 225 and the power subsystem 228. The sensor subsystem 224 generally captures data local to the worker, the communications subsystem 226 communicates this data to the monitoring subsystem 240 and the BAS executive subsystem 221 provides configuration and other features to the BAS 220. The communications subsystem 226 also receives data from the monitoring subsystem 240 such as alerts and status data. The sensor module user interface 225 provides information to the user either through patterns or colors of lights or through a small display. The power subsystem 228 is configured to power the components of the sensor package or an attached component block.

In some embodiments, the component block 260 may be modular component blocks. For example, in some embodiments, the component block 260 may be an external battery and the power subsystem 228 would draw power from the battery component block to provide power to other components. And in some embodiments, the component block 260 may be an additional sensor to provide additional sensor data. And in some embodiments, the component block 260 may be an antenna.

In some embodiments, the status band 230 generally provides a local status to the worker. Status may be received by the receiver 234. Through a status band user interface 232, the status band 230 may communicate alerts to the worker and may allow the worker to provide additional data to the monitoring subsystem 240.

In some embodiments, the BAS 220 may communicate with the monitoring subsystem 240, to share information between all other components, save data, and preserve system state.

In some embodiments, the web client applications generally allow for storage of data and allow for remote status and remote management of the CSS, CSS subsystems, algorithms, and data.

### Sensor Module and Sensor Subsystem:

Referring to FIG. 2A, the BAS sensors in the sensor subsystem 224 are generally selected to provide data such as, but not limited to: (1) location data, (location in comparison to a known layout or floorplan or location data as geocoordinate information); (2) environmental data; (3) physiological data; (4) behavioral data, and (5) physical data such as body orientation or posture data.

Referring to FIG. 2A, the software and hardware architecture for the sensor module 280 is designed to support the hardware modularity. Typically, the initial sensor subsystem 224 is housed in the sensor module 280. The sensor module 280, also referred to as a packaged sensor, supports multiple types of modularity by: (1) changing the optical sensor components and (2) adding a different component blocks 260. The sensor subsystem software architecture will identify the component(s) connected and perform the appropriate processing. This can be accomplished with a containerization approach, such as with sensor modules and component block, where sensor modules can be swapped out depending on the need and each sensor component has an associated component block that can be added to the CSS to perform the function of that component.

Referring to FIG. 2A the sensor module 280 hardware is packaged to support modularity within a common sensor profile. For the two types of modularity, an optical sensor hardware component uses light-based technologies for physiological measures and can be used with a sensor module, and other component blocks can provide other sensing technologies—such as but not limited to atmospheric sensing, chemical sensing, biological sensing, radiological sensing via electrochemical, thermal sensing, gravimetric, acoustic, etc. sensing technologies.

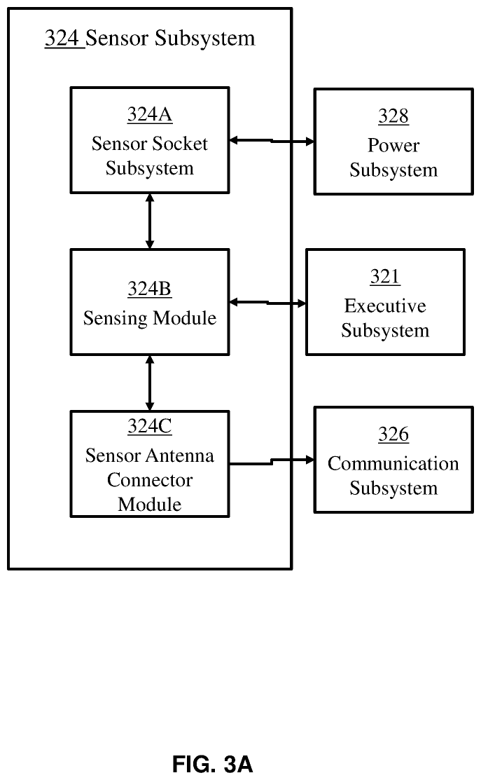

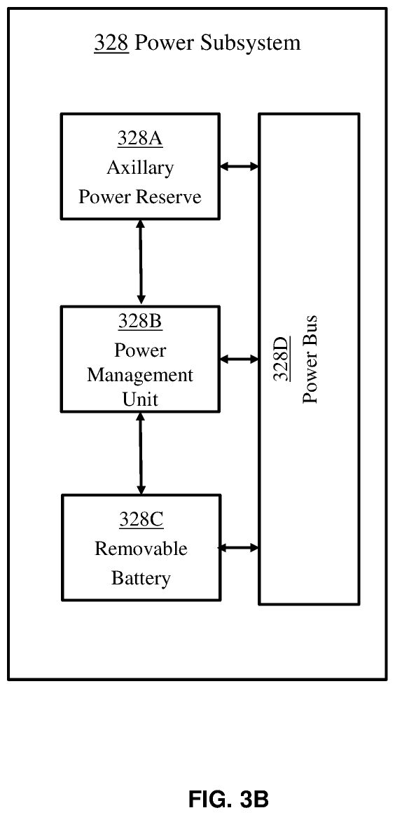

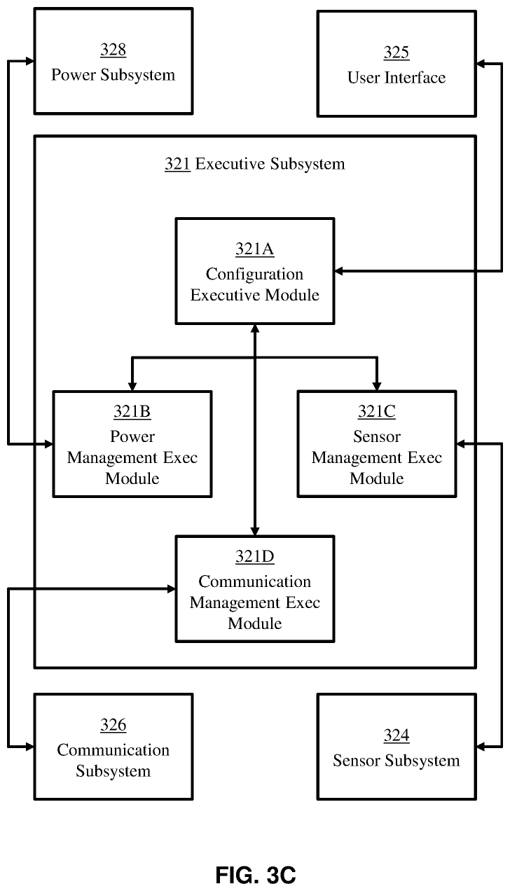

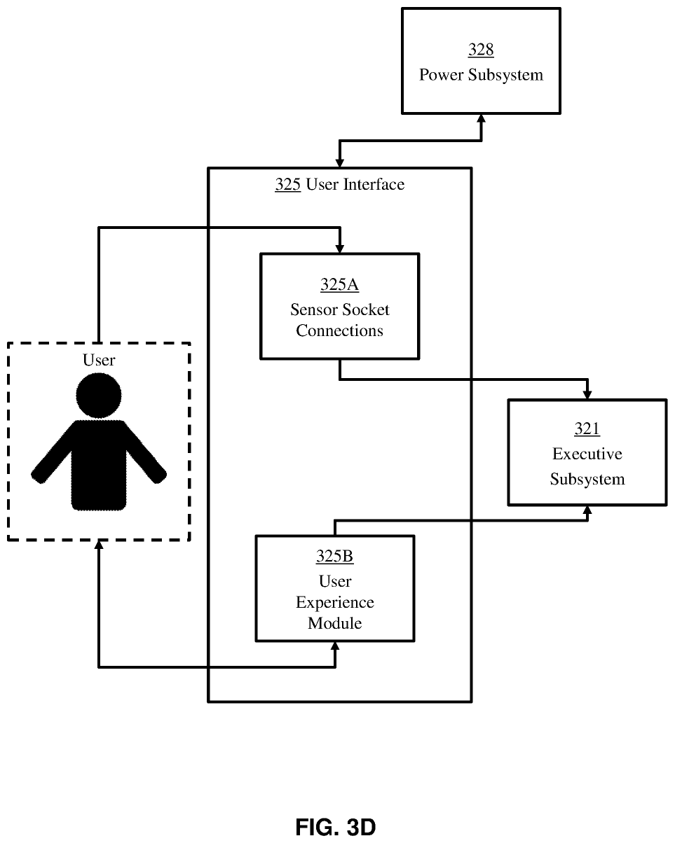

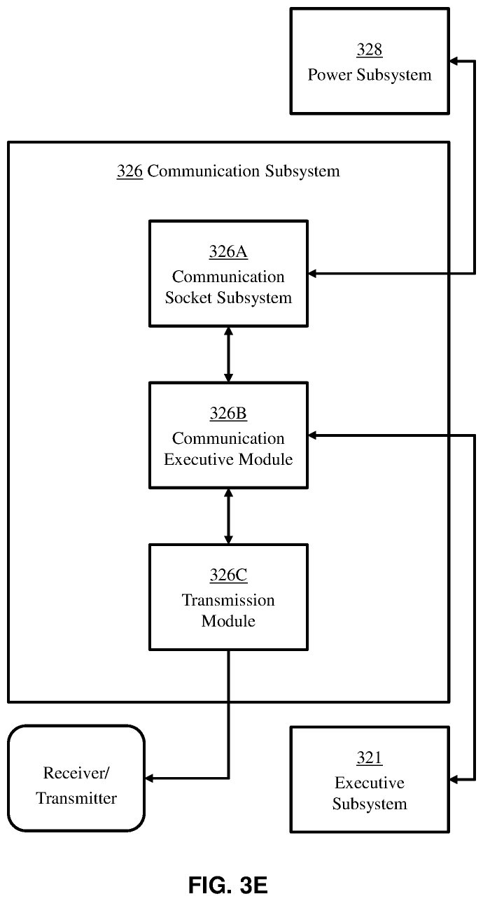

FIG. 3A illustrates details of an example sensor subsystem 324 as part of a sensor module. The example shows a sensor subsystem 324 with interconnected components comprising a sensor socket subsystem 324A, a sensing module 324B and a sensor antenna connector module 324C. The sensor socket subsystem 324A may be connected to the power subsystem 328, the sensing module 324B may be in communication with the executive subsystem 321 and the sensor antenna connector module 324 may be in communication with the communication subsystem 326.

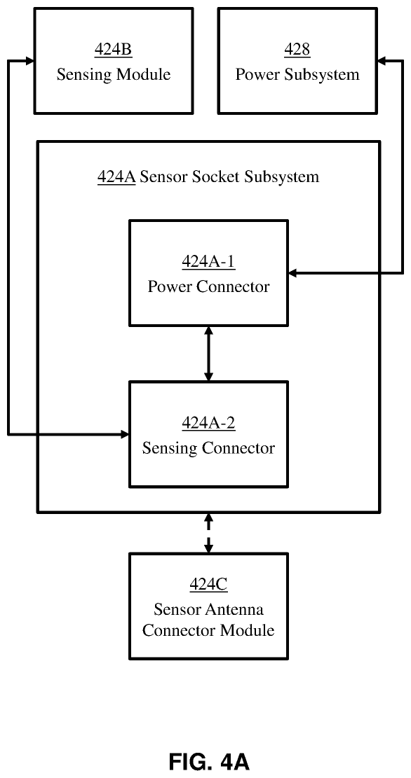

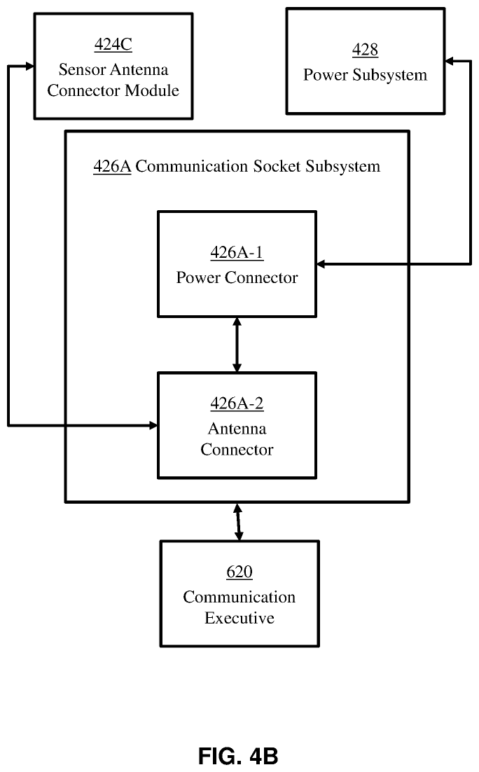

FIG. 4A illustrates details of a sensor socket subsystem 424A in communication with the sensor antenna connector module 424C. The sensor socket subsystem 424A comprising a power connector 424A-1 connected to the power subsystem 428 and a sensing connector 424A-2 in communication with the sensing module 424B.

### Power Subsystem:

Referring to FIG. 2A, the power subsystem software architecture is designed to support modularity by providing the power necessary for the sensing module, optical sensing component and sometimes component blocks. The power subsystem software architecture provides power to the coupled sensing components. Additionally, the power subsystem software will detect when a battery component block is coupled in order to route the power from the battery to other components.

Referring to FIG. 2A, the power subsystem power system hardware supports the modularity of components via a physical connection to the components which is capable of establishing if a connected component is drawing power or providing power.

FIG. 3B illustrates details of an example power subsystem 328 having interconnected components comprising an axillary power reserve 328A, a power management unit 328B, a removable batter 328C and a power bus 328D.

### Executive Subsystem:

Referring to FIG. 2A, the executive subsystem software architecture supports modularity since it controls the flow and timing of the data from the connected components. With the containerized software approach of the other subsystems the executive subsystem is designed to detect and communicate with the other subsystem components to facilitate the flow between the components.

FIG. 3C illustrates details of an example executive subsystem 321 comprising a configuration executive module 321A, a power management executive module 321B, a sensor management executive module 321C and a communication management executive module 321D. The configuration executive module 321A may be in communication with a user interface 325, the power management executive module 321B is connected with the power subsystem 328, the communication management executive module 321D may be in communication with the communication subsystem 326 and the sensor management executive module 321C is in communication with the sensor subsystem 324.

### User Interface:

The CSS uses physical objects—such as COTS sensors and sensor components—as input devices (i.e., sensors to provide data display mechanisms) and output devices (e.g., display mechanisms). The solution may incorporate existing physical objects, however, it also uses these objects to produce numerous new features. One example is the use of a smartwatch, which uses reflectance-based pulse rate monitors and an onboard accelerometer to provide estimated heartrate and motion. The smartwatch also serves as a multi-modal alerting/display mechanism by providing auditory, visual, and haptic alerts to the human worker if dangerous conditions or activities are detected.

Referring to FIG. 2A, the architecture of the sensor module user interface 225 supports modularity in that the individual sensor module user interface 225 for either the sensor module 280 or the component blocks 260 are able to display information to the user. The architecture supports a standard set of communication and notification signals which is supported by the hardware of those components.

FIG. 3D illustrates details of a sensor module user interface comprising a sensor socket connections 325A and a user experience module 325B. The sensor module user interface 325 is connected with the power subsystem 328 and the sensor socket connections 325A and the user experience module 325B is in communication with the executive subsystem 321.

### Communication Subsystem:

Referring to FIG. 2A, the BAS communication subsystem 226 generally provides the communications link between the BAS and the monitoring subsystem. In some embodiments, the BAS receiver/transmitter 234 is configured with wireless connectivity to provide the ability to connect and live stream each worker's sensor data to the monitoring subsystem elements such as decision support station displays. This prevents the server from having to manage sensor connections directly, making the CSS more scalable. Additionally, because the BAS can remotely connect to the server (via WiFi or cellular network), users do not need to stay within a certain physical range of the server (making them more mobile). Another benefit of the BAS receiver/transmitter is that it runs as a background service. This ensures the application is always running and maintaining an active connection with the server. It also limits user interaction with the system, which allows users to focus on their tasking without distraction.

Location data supports the identification and tracking of workers while in the working area. This allows for the development of derived metrics, such as establishing both worker location and length of time within one location.

The communication subsystem software architecture supports modularity, by having a capability to identify and use the communications protocols available with the connected components. For example, a sensor module may only contain a Bluetooth communication capability to provide short range transmission of data the BAS. However, when a component block with a LoRa communication capability, the communication subsystem architecture will use the communication protocols available. In the case of LoRa this will allow long distance communication to a receiver. The communication subsystem hardware needs to be able connect with the attached components to provide data to the transmission electronics for the protocol. The communication subsystem provides the available sensor data to allow the communication mechanisms for the Bluetooth and LoRa to transmit the data.

FIG. 3E illustrates details of an example communication subsystem 326 comprising a communication socket subsystem 326A, a communication executive 326B and a transmission module 326C. The communication socket module 326A is connected to the power subsystem 328, the communication executive module 326B is in communication with the executive subsystem 321 and the transmission module 326C is in communication with a receiver/transmitter.

FIG. 4B illustrates details of an example communication socket subsystem 426A in communication with a communication executive 620. The comprising a power connector 426A-1 and an antenna connector 426A-2. The power connector 426A-1 is connected to the power subsystem 428 and the antenna connector 426A-2 is connected to the sensor antenna connector module 424C.

### Monitoring Subsystem:

The monitoring subsystem may comprise systems as disclosed in U.S. Pat. Pub. No. 2022/0277254, published on Sep. 1, 2022, which is herein incorporated by reference in its entirety.

The monitoring system may be configured to send and receive large amounts of data from different users simultaneously via the message broker. The primary source of data comprises the connected users' BAS which consists of which may include (1) location data, (2) environmental data, (3) physiological data, (4) behavioral data, and (5) physical orientation/posture data. The data received may be raw data or it may be pre-processed by the BAS.

This data is fed to the expert subsystems that fuse, or contextualize, the data to determine the contextualized state, the contextualized environment and in some embodiments, the contextualized situation. The expert subsystem may comprise a series of predefined rules defined based on a long series of information synthesis from literature, interviewing subject matter experts, and research findings in data collected. The rules may be stored in the subsystem database and allow for a determination of contextualized worker states such as the worker being in a compromised state or trending towards a compromised state. Similar rules for the environment may allow for a determination of a contextualized environment such as high levels of CO which may be acceptable in one space but not acceptable in another. Together, the contextualized worker state and contextualized environmental state may provide a contextualized situation.

Furthermore, the monitoring subsystem may comprise decision support modules that provide decision support to the worker and CSS user. These decision support modules may enable safety attendants to readily monitor maintainer/worker health and safety while identifying hazards with low false alarm occurrences.

### Modularity:

The sensor module, the mounting base and the component block are configured to support modularity. Generally, this modularity is provided by a both having electronic connections were signals, data, and power can be transmitted between the components.

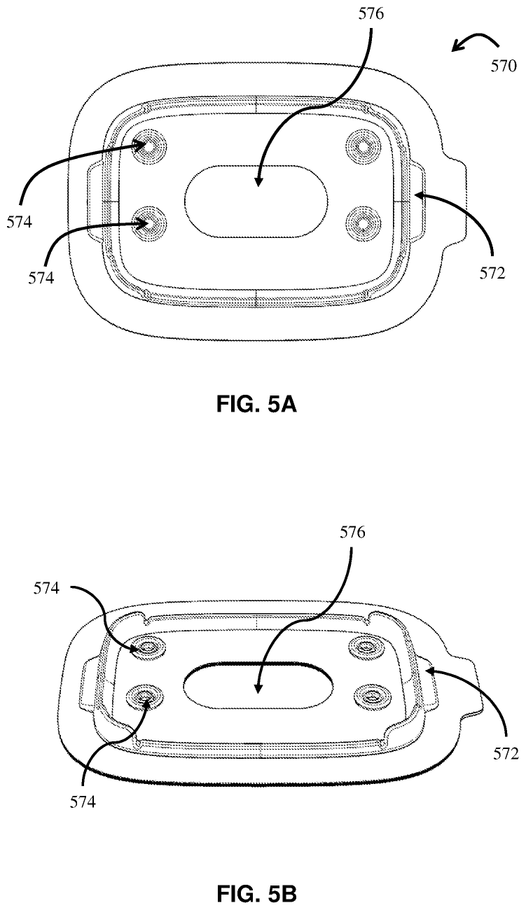

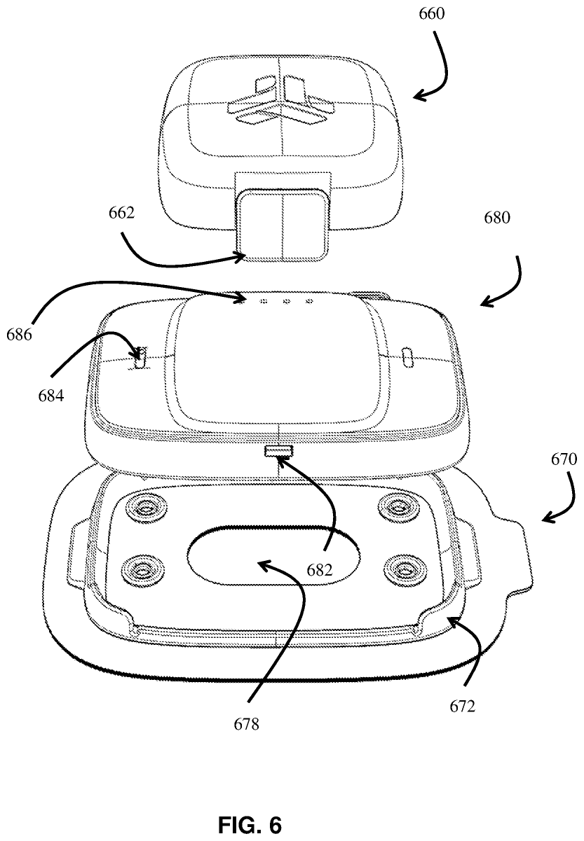

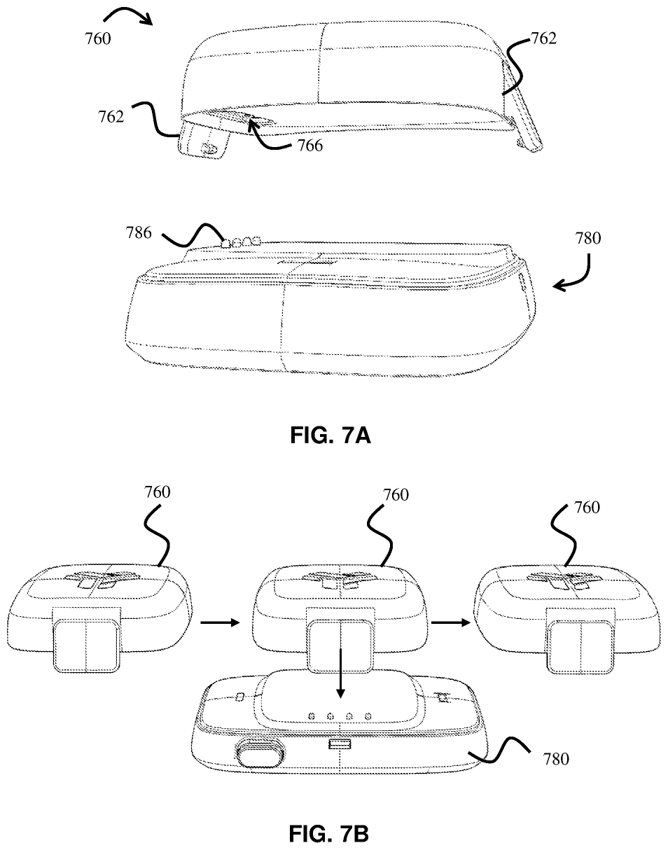

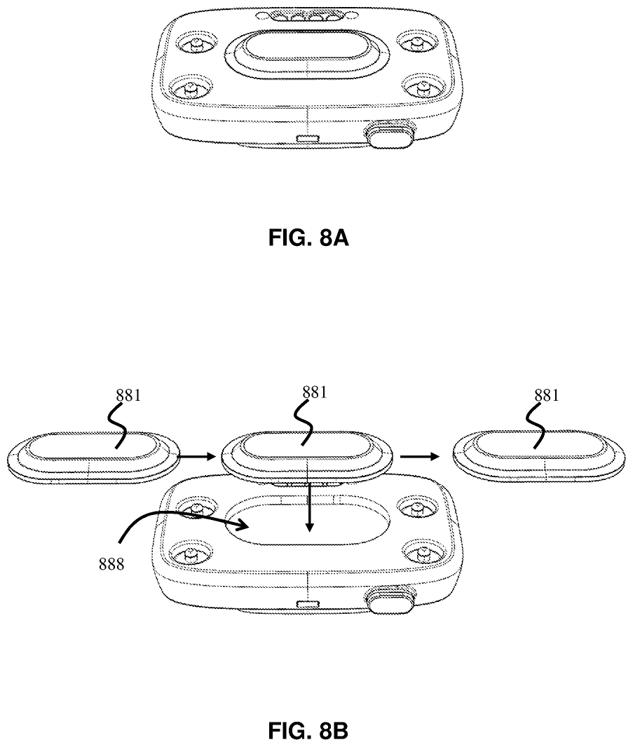

The following describes each of the modular components and their modularity in detail referring to the hardware FIGS. 5A, 5B, and 6 shows the modularity of sensor module with mounting base using the mechanical clasps to secure the sensor module to the base. Referring to FIGS. 6, 7A, 7B shows the modularity of component blocks with sensor module using the mechanical clasps. Referring to FIGS. 8A and 8B shows the modularity of optical sensor with sensor module.

As shown in FIGS. 5A and 5B, the mounting base 570 comprises a base coupling element 572, such as a pliant mechanical clasp, to secure the package sensor to the mounting base 570, electrical sensor contacts 574, and through hole creating a mounting base optical window 576. The mounting base 570 is configured to removably mate with other CSS components such as component blocks with coupling elements.

FIG. 6 illustrates an example of how CSS components may be configured to removably mate with each other. As shown, the CSS components comprise the mounting base 670, a sensor module 680 and a battery component block 660 to function as a secondary battery. The battery component block 660 connects to the sensor module 680 secured by coupling elements on both blocks. For example, the clasps 662 on the battery component block 660 are pliant and configured to couple with a clasp recess 682. The sensor module 680 is shown to have user interface status lights 686 and connectors 684, being secured to the mounting base 670 with the base pliant clasps 672, allow the sensor module 680 access to the mouthing plate optical sensor window 678.

As shown in FIG. 7A a secondary battery or battery component block 760 is shown to connect to a sensor module 780 via the mechanical clasps 762. Also shown are electrical connectors 766 configured to mate with electrical connectors 786 of the sensor module 780.

As shown in FIG. 7B, component block switching is shown with multiple instances of component blocks 760 to couple to the sensor module 780.

As shown in FIGS. 8A, an optical component may be inserted into the sensor module to allow the sensor to perform light-based sensing as a sensor module.

As shown in FIG. 8B, multiple instances of an optical component 881 is shown to demonstrate how they would be inserted and removed from the optical component window 888 within the sensor module hardware.

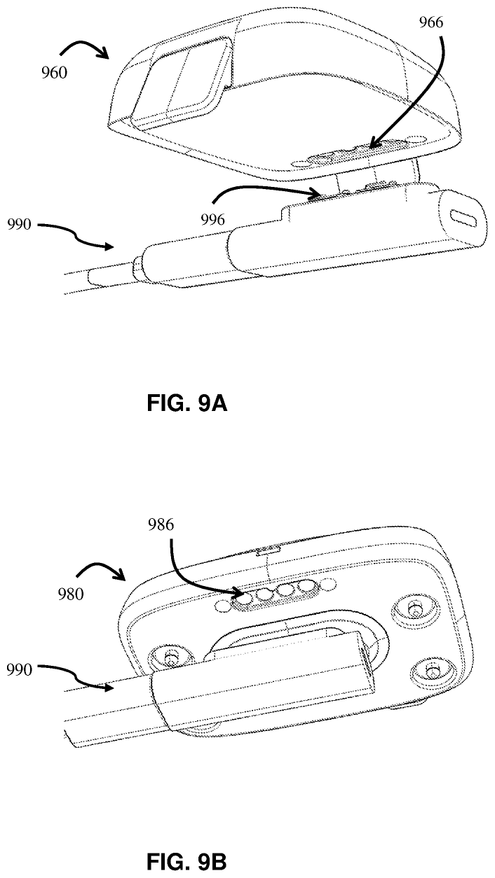

As shown in FIG. 9A, a magnetic charger 990 with POGO pin connectors 996 may be used to supply power to charge a component block 960 with electrical connectors 966. FIG. 9B shows the magnetic charger 990 with POGO pin connectors configured to supply power to charge a sensor module 980 with the electrical connectors 986. The single cable and common charger design to charge any component block or module facilitates the modularity of components.

The modularity of the components allows for the contextualize sensor system to be configured to address multiple situations with a common design. For example, if the sensor system is used in fuel tank the system would be configured with a gas sensor. This specifically allows for the sensor to measure gases that pose a danger to the user. Danger could include but not limited to oxygen levels being low to impact the workers ability to work, oxygen levels too high as to be an explosion risk, unsafe carbon monoxide levels. As another example, if the sensor system is in an industrial area where toxic industrial chemical may pose a threat to workers, systems would be configured with sensors with high sensitivity and selectivity to the chemicals of interest. The modularity also allows teams of workers extend the coverage of toxin detection. For example, one worker to be configured to detect gases such as ammonia and chlorine while another worker is configured to detect hydrogen sulfide and hydrogen fluoride all monitored by a common monitoring platform. In this case each member of the team would target different toxin of interest instead of all members required to wear redundant sensors which could prove difficult especially within a confined space where space is limited.

### Processor Based Embodiments of the Contextualized Sensor System

The presented solution improves processor-based systems that attempt to remotely monitor via computer-based technology, in particular monitoring of workers in unconventional postures and spaces. The addition of context to health status alerts allows for remote systems, or a human remote attendant, to use contextualized data for optimal decision making in times of stress. The solution also minimizes the number of false positive alarms seen in similar systems, which undoubtedly contribute to at least a minimal amount of alarm fatigue.

As will be readily apparent to those skilled in the art, contextualized sensor systems and methods of using them can be embodied in hardware, software, or a combination of hardware and software. For example, a computer system or server system, or other computer implemented apparatus combining hardware and software adapted for carrying out the methods described herein, may be suitable. One embodiment of a combination of hardware and software could be a computer system with a computer program that, when loaded and executed, carries out the respective methods described herein. In some embodiments, a specific use computer, containing specialized hardware or computer programming for carrying out one or more of the instructions of the computer program, may be utilized. In some embodiments, the computer system may comprise a device such as, but not limited to a digital phone, cellular phone, laptop computer, desktop computer, digital assistant, server or server/client system.

Computer program, software program, program, software or program code in the present context mean any expression, in any language, code or notation, of a set of instructions readable by a processor or computer system, intended to cause a system having an information processing capability to perform a particular function or bring about a certain result either directly or after either or both of the following: (a) conversion to another language, code or notation; and (b) reproduction in a different material form. A computer program can be written in any form of programming language, including compiled or interpreted languages, and it can be deployed in any form, including as a stand-alone program or as a module, component, subroutine, or other unit suitable for use in a computing environment.

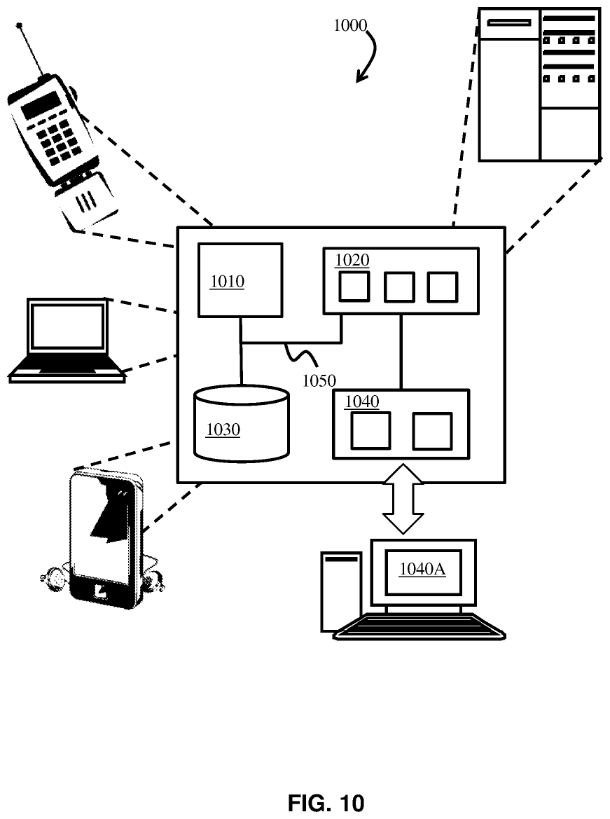

FIG. 10 is a schematic diagram of one embodiment of a computer system 1000 by which the contextualized sensor system methods may be carried out. The computer system 1000 can be used for the operations described in association with any of the computer implemented methods described herein. The computer system 1000 includes at least one processor 1010, a memory 1020 and an input/output device 1040. Each of the components 1010, 1020, and 1040 are operably coupled or interconnected using a system bus 1050. The computer system 1000 may further comprise a storage device 1030 operably coupled or interconnected with the system bus 1050.

The processor 1010 is capable of receiving the instructions and/or data and processing the instructions of a computer program for execution within the computer system 1000. In some embodiments, the processor 1010 is a single-threaded processor. In some embodiments, the processor 1010 is a multi-threaded processor. The processor 1010 is capable of processing instructions of a computer stored in the memory 1020 or on the storage device 1030 to communicate information to the input/output device 1040. Suitable processors for the execution of the computer program instruction include, by way of example, both general and special purpose microprocessors, and a sole processor or one of multiple processors of any kind of computer.

The memory 1020 stores information within the computer system 1000. Memory 1020 may comprise a magnetic disk such as an internal hard disk or removable disk; a magneto-optical disk; an optical disk; or a semiconductor memory device such as PROM, EPROM, EEPROM or a flash memory device. In some embodiments, the memory 1020 comprises a transitory or non-transitory computer readable medium. In some embodiments, the memory 1020 is a volatile memory unit. In another embodiment, the memory 1020 is a non-volatile memory unit.

The processor 1010 and the memory 1020 can be supplemented by, or incorporated in, ASICs (application-specific integrated circuits).

The storage device 1030 may be capable of providing mass storage for the system 1000. In various embodiments, the storage device 1030 may be, for example only and not for limitation, a computer readable medium such as a floppy disk, a hard disk, an optical disk, a tape device, CD-ROM and DVD-ROM disks, alone or with a device to read the computer readable medium, or any other means known to the skilled artisan for providing the computer program to the computer system for execution thereby. In some embodiments, the storage device 1030 comprises a transitory or non-transitory computer readable medium.

In some embodiments, the memory 1020 and/or the storage device 1030 may be located on a remote system such as a server system, coupled to the processor 1010 via a network interface, such as an Ethernet interface.

The input/output device 1040 provides input/output operations for the system 1000 and may be in communication with a user interface 1040A as shown. In one embodiment, the input/output device 1040 includes a keyboard and/or pointing device. In some embodiments, the input/output device 1040 includes a display unit for displaying graphical user interfaces or the input/output device 1040 may comprise a touchscreen. In some embodiments, the user interface 1040A comprises devices such as, but not limited to a keyboard, pointing device, display device or a touchscreen that provides a user with the ability to communicate with the input/output device 1040.

The computer system 1000 can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include, e.g., a LAN, a WAN, wireless phone networks and the computers and networks forming the Internet.

One example embodiment of the contextualized sensor systems and methods of use may be embodied in a computer program product, the computer program product comprising a computer readable medium having a computer readable program code tangibly embodied therewith, the computer program code configured to implement the methods described herein, and which, when loaded in a computer system comprising a processor, is able to carry out these methods.

### One Embodiment of Methods to Contextualize Sensor System Data

For illustration purposes and not for limitation, one example embodiment of methods of using the CSS generally comprise receiving sensor data from one or more sensor, fusing the data to provide some contextualized data, comparing the contextualized data to an alerting library to determine whether an alert situation has occurred and if the alert situation has occurred, communicating an alert through a user interface. The sensors may comprise an environmental sensor, a location sensor, a physiological sensor, a behavior sensor and a posture sensor, and the sensor data may comprise an environmental data, a location data, a physiological data, a behavior data and a posture data.

Because of the modularity of the CSS components, different component blocks with different sensing capabilities may be combined to provide multiple sensing capabilities to reflect the situation the CSS will be used in.

In some embodiments, the contextual sensor system collects physiological data to also help assess cognitive readiness of the worker.

### Example Embodiments of the Contextualized Sensor System in Operation

Operation of one embodiment of the contextualized sensor system is described below by outlining the steps, methods, and components that are required from process origin to completion.

The first step requires the instrumentation of the human and the environment. The human to be monitored will be equipped with the following sensors and devices: (1) a sensor-embedded garment or strap for cardiopulmonary monitoring, (2) a wearable or hand-held atmospheric monitor, which is equipped to sense pre-identified gas concentration (e.g., oxygen or volatile organic compounds), (3) a personal location tracking sensor unit or “puck”, (4) a smartwatch, and (5) a smartphone, though this device typically only need to be placed in the general vicinity of the human.

The environment also needs instrumented with location beacons (e.g., BLE- or UWB-enabled) to enable real-time location tracking in GPS-denied environments.

After the human operator has been equipped, the sensors will be turned on to ensure that the requisite data for optimal system utility is available. These data sources will then be fused to a centralized unit, which is colloquially referred to as a body area or personal area subsystem (BAS or PAS). The BAS for the presented application is currently a rugged smartphone due to their optimal processing power and various innate methods for wireless communication. While a smartphone currently does a satisfactory job of serving as a BAS, the system in operation could use other devices (e.g., LTE-enabled smartwatch), especially if the wearable technology industry and internet of things (IoT) initiative continues to progress.

Though some onboard processing occurs within the aforementioned sensors and components, there is a fair amount of data processing that still must occur. As such, the data is then transmitted via a major cellular infrastructure (e.g., Verizon Wireless LTE) to a cloud-based web server (e.g., Amazon Web Services [AWS]).

The next steps “Processed Real-Time Signals”, “Data Fusion for Contextualized Data”, and “Health & Safety Hierarchical Alerting Library” all may occur within the cloud-based web server.

First, the data are partitioned into 1-second queues. This step ensures that all data sources are temporally aligned—since many of the devices and subsequent data streams have disparate sampling rates.

The data streams are then used in isolation and concomitantly to provide context about the situation of the human in applied setting. A good example of this is the fusion of heart rate and accelerometer data. A higher-than-normal heart rate (e.g., 170 beats per minute [bpm] compared to a resting baseline of 70 bpm) can really only be labeled problematic if the observer has additional context about the situation. For example, this heart rate would be normal for an individual undergoing cardiovascular exercise. However, this heart rate would be abnormal for an individual doing work activities that require little or no strenuous exercise (e.g., putting in rivets in a supine position). This is where behavioral, location, and postural data can provide additional context.

The fusion of behavioral, location, and postural data sources can generally provide derived estimates of human activity through limited data analysis. For example, someone in an upright position, moving at a speed of about 1-1.5 m/s, and outside of a confined space, is likely walking based on a general understanding of the situation and normal human anatomy and physiology. In this instance, a slightly elevated heart rate is to be expected. However, if the data sources and associate analysis indicate that someone is laying on their side (i.e., recovery position) or front (i.e., prone), displaying little or no movement, inside of confined space, but presenting significantly elevated heart rate, there is likely an issue with the individual or the physiological monitoring equipment. Both of these contrived conditions would necessitate a prompt response from the local support staff. These instances provide an example of the “Alert Determination” where the data are fused with a pre-existing health and safety alerting architecture or library.

The “Health & Safety Hierarchical Alerting Library” houses pre-defined rules for health and safety alert generation. Most of these rules are based on combination of expected values (e.g., baselines), past data, normal physiology and behavior, and context. Some of the specific alerts housed in this library include: (1) sensor and network disconnects, (2) anomalous heart rate values and patterns, (3) anomalous respiration patterns, (4) detection of breathing cessation, (5) anomalous core body temperatures (both high and low), (6) anomalous atmospheric composition, and (7) low battery warnings. While non-exhaustive, this list provides a snapshot of the current alerting paradigm. Furthermore, it should also elucidate the relationship between raw sensor data and data context to allow for optimized alerting.

Lastly, the contextualized data and subsequent alerts then need to be communicated to the appropriate personnel. For the presented solution, these alerts-which are customized by job function—are transferred via user interfaces (UIs) to the human in the unconventional space or posture via smartwatch, to a roving (i.e., ground-based) attendant via tablet, and to a central monitoring station via desktop. While the specific methods for alerting may fall outside of the scope of this disclosure, the mechanism does not. These contextualize alerts can be fed to BLE- or internet-enabled hardware devices, such as tablets, smartwatches, or desktop computers.

In summary, the claimed invention includes a fusion of multiple data sources with contextual information to optimize the accuracy of alerts, which will ultimately be displayed on a hardware device. In other words, the fusion of data and context allows for more accurate and detailed information as to whether a dangerous incident is likely to ensue or currently underway.

This claimed innovation currently resides in a confined space monitoring system, though the fusion of physiological data, environmental data, location data, behavioral data, and postural data to provide context-enabled health and safety alerting could be applicable to various human monitoring applications.

### Additional Example Application, Automated Maintainer (Worker) Health Status Classification

An automated, real-time health status assessment capability for each maintainer (i.e., worker) utilizes a set of model-based classifiers that derive an estimate of maintainer health status by processing the various data sources collected by CSS. Data inputs to a health status classifier may include: physiological signals (heart rate, respiration); atmospheric levels (O₂, LEL, VOC); behavioral indicators (e.g., degree of physical movement); and physical location (e.g., inside a confined space).

The output of the health status classifier is a discrete state indicating one of three possible states a maintainer is in: optimal, sub-optimal, and emergency. An “optimal” state indicates that the subject is in no foreseeable risk of suffering an adverse outcome, and the majority of data sources are within desirable levels and not trending toward undesirable levels. A “sub-optimal” state indicates that one or more data sources are trending toward undesirable levels. Although a sub-optimal state does not constitute an emergency situation, the affected maintainer should be monitored more closely, and preventative intervention may be needed to ensure the maintainer's health/safety does not further degrade. An “emergency” state indicates that one or more data sources have exceeded desired levels, and that immediate intervention is required and justified, such as immediately vacating the affected maintainer from a confined space and, if needed, contacting EMS. These three possible health states may be continuously fed to the decision support station. They are represented with a “stoplight chart” for each maintainer, in which an optimal state is green, sub-optimal state is yellow, and emergency state is red. The automated health status classifier provides the amount of time a maintainer has been within each state since this information is critical to determining if and when an intervention is needed.

Two additional states were identified that are not specifically health related yet remain important to properly prioritize the state of a maintainer. One is a disconnection status—this can refer to either a sensor disconnection (e.g., out of range, out of battery) or BAS disconnection from the CSS server (e.g., application crashes, Android device shuts off). In both cases, the same end result ensues in that the affected maintainer cannot be safely monitored remotely. Therefore, rapidly identifying these cases is of utmost importance. The maintainer disconnect status is color-coded as an “orange” state. The other case is that of a non-urgent (not safety critical) request for service, which is color-coded as a “blue” state. Service requests are typically (though not exclusively) initiated manually in situations when a maintainer needs help and it is not a matter implicating health and safety, such as needing a tool retrieved, requiring information, and receiving approval to enter a confined space.

Three distinct data processing layers were developed to facilitate the automated health status classification requirement for CSS: (1) signal processing; (2) data fusion; and (3) expert system. Signal processing refers to the conversion of raw signals into usable data features; it is a general term that can refer to various functions such as filtering noise, managing data volume, and refining data features before they are used in other system layers. The majority of signal processing in the CSS prototype is implemented on-board the wearable sensors and by BAS software on the Android device. Data fusion refers to the integration of multiple disparate measures to improve assessment capabilities, thereby better perceiving the “complete picture.” The expert system refers to alerting decision rules that flag potential existence of health/safety problems, while balancing with false alarm tradeoffs. The majority of data fusion and expert system functions occur on the CSS server.

The expert subsystem module comprises the situation classifier algorithms and table that monitor and apply each maintainer's data to classify health status as one of the three discretized states (optimal, sub-optimal, and emergency). The health status classifier defines thresholds and boundaries that are identified as sub-optimal, and with a certain level of severity, by monitoring the incoming data sources with respect to predefined values, thresholds, and ranges of values. Data sources are continuously monitored across the available categories of data (physiological, environmental, behavioral). An initial set of acceptable values, thresholds, and ranges for each variable was defined with a qualified multi-disciplinary team of healthcare experts, biomedical engineers, and human factors/safety specialists, to name a few. These parameters were selected with the expressed intention to maximize opportunities to detect health and safety incidents, while minimizing the likelihood of false alarms. Subsequently, high-fidelity testing data was collected to further refine and optimize these parameters. The expert system underwent iterative modification throughout the project and, by the conclusion of the RIF effort, demonstrated the ability to effectively balance the need to alert legitimate issues (e.g., no-motion detected, excessively high heart rate over long durations, breathing cessation) while encountering a low false alarm rate.

As part of this objective, an automated physiological baseline collection capability was implemented. This runs as an autonomous background service that examines recent windows of data to find sustained lower values using a Savitzky-Golay filter. Currently this processing routine is applied to heart rate data only, but it can be applied to any data feature as deemed necessary. The purpose in collecting baseline data is to allow a more individualized means for evaluating a person's data—i.e., relative to their baseline state. More specifically, the system computes “difference from baseline” metrics that can be used as the basis for health status classification and alerting. It is important to note the baseline capabilities assume an individual is healthy prior to entering a confined space.

Importantly, the CSS Gen1 sensor suite had to be defined and implemented to interoperate effectively with the worker health classifiers. Although the atmospheric sensor was essentially pre-selected to be the RAE Systems MultiRAE (O₂, LEL, VOC detection) based on prior LM-Aero experience and limited COTS alternatives being available, the COTS physiological sensor options were far more expansive. There were two primary selection criteria:

The physiological sensor suite must provide adequate quality of data to enable early warning and emergency detection of hazardous worker health states.

The sensors must offer a form factor that is comfortable, does not interfere with worker work, carries acceptable cost, and is sufficiently user-friendly to use.

To make a final decision, a wide range of cardiopulmonary wearable sensors were tested. Although some sensors are comparable in terms of cost and performance, the final down selections were made by allowing workers to view and try on inert sensors while crawling into non-permit confined spaces. This exercise led to the clear decision of using the Polar Team Pro electrocardiogram (EKG) base layer shirt, Polar H10 health sensor, and Samsung Galaxy Watch. Cardiopulmonary signals can be acquired by the Polar H10 when inserted into the Polar Team Pro shirt, while motion data can be acquired from the Galaxy Watch. As shown in the image below, the Galaxy Watch responded to Safety requests and end user preferences by mounting the watch unit into an arm band form factor. Of note, the Zephyr Bioharness remains available and fully compatible with this sensor suite (i.e., worn over the Polar Team Pro shirt for more robust respiratory data), but did not meet end user preferences due to comfort level and obtrusiveness.

### Additional Example Application, Decision Support Station

One example application is to use the CSS to provide a decision support station that provides reliable detection of problematic events with a low false alarm rate. This application moves away from the current 1-to-1 ratio of standby attendants per every person in a confined space, which can improve manpower utilization and results in significant cost savings and accelerated maintenance schedules. However, employing a “many-to-one” ratio of confined space monitoring requires an operational role that is dedicated exclusively to monitoring worker health and safety. This Remote Safety Attendant role—referred to as the RSA—will ultimately be a main user of CSS.

A CSS decision support station must support the RSA's ability to coherently understand worker health and safety indicators, determine if and when a serious situation has (or is about to) occur, and initiate a timely intervention that is appropriate for the situation. To this end, the decision support station may convey four categories of real-time sensor data: physiological indicators; atmospheric hazards detected in the environment; worker locations; and behavioral activity detected by worn accelerometers. The overall health of each worker must also be clearly available from the health classifier algorithms (technical objective #2).

The decision support station includes a collection of informational displays and UIs spread across multiple computer screens. In one embodiment, the decision support station design includes a map-based display to intuitively communicate worker locations within a single view. Visual overlays (e.g., shape, color) may be used to convey metadata for each worker. The UI is sufficiently flexible to scale up to a large number of workers to accommodate the size of ALC operations. Due to the large number of workers to be monitored by a single RSA, the station design incorporates system alerting logic and “tripwires” to ensure potential issues are highly salient to RSAs. For example, if a worker's breathing rate drops below a certain rate per minute, the RSA will be alerted to this promptly. Alerts are delivered to the station both visually and through the use of auditory cues to reinforce these alerts. The ability to combine these system features under the decision support station ensures reliable detection is always present.

This embodiment may also be configured to provide a low false alarm rate. Although occasional false alarms are expected and impossible to prevent altogether, a low false alarm rate may be achieved by introducing a multi-modal sensor suite and cross-sensor data fusion to diversify how alerting decisions are made. More specifically, this is accomplished by the fact that if one sensor type fails or encounters an anomaly resulting in a false alarm, the other sensors and data sources are present to prevent the false alarm. For example, if breathing rate is suddenly not detectable for a worker due to a sensor malfunction, the presence of other sensor data such as heart rate, movement, location, and atmospheric levels provide a clearer picture of the cause. The automated data fusion provided by the health status classifier greatly helps with this interpretation of the data. Fast and easy recovery from false alarms is also key, which is accomplished through the station's UI design. The primary alerts information and resolution functionality is available to the RSA through a primary overview page. This UI also includes additional utility by automatically organizing all active workers according to their active work area and by their confined space entry status (i.e., not approved to enter, approved to enter, in confined space).

A worker status UI may be configured to specifically to answer these questions in a fast and user-friendly manner, effectively using a dial graph for each question. Abnormal states are highly salient and alerts are readily viewable when they occur. Furthermore, the aforementioned worker disconnections (i.e., orange alerts) and service requests (i.e., blue alerts) are also rapidly identified through this UI. If an even more detailed look at the data is desired, the status UI may include an additional tab labeled “Feature Data” in which any data feature available for the selected worker can be viewed at any time and at any time scale (up to the past one hour). Further yet, data that spans beyond one hour in the past can be exported through a web-based data export tool and easily filtered and plotted through a third-party application (e.g., Excel).

Another aspect of the decision support station involves the electronic entry request sub-system. This sub-system ensures that workers are not allowed to enter a confined space until the RSA has acknowledged their request to enter. This is intended to ensure the worker's sensor data is visible and fully functional prior to entry, as well as to ensure the RSA is provided situational awareness regarding each worker's purpose for entry, time of entry, and approximate location. Each worker initiates this process by first entering atmospheric samples into a web-based UI for all confined spaces to be entered. This is followed by completing and submitting a confined space entry form. The entry form and corresponding atmospheric samples for the applicable confined spaces are made available to the RSA for review, at which point the RSA can approve the request to enter.

### Additional Example Application, Early Detection and Coordination with EMS Teams

In some embodiments, the CSS is configured to support interventions to ensure worker health/safety through early detection and coordination with EMS teams. These capabilities are desired to increase the likelihood of preventing serious problems, and to accelerate response times when an intervention is needed. CSS provides tools, information, and functionality that leverage system capabilities for early interventions that serve as preventative measures to reduce the progression to more serious incidents.

To determine if and when a particular intervention is needed, CSS relies on health status classifiers and the decision support station to help the RSA decide if/when a worker has reached or is trending toward an undesired state. A “sub-optimal” (i.e., yellow) classification indicates that risk has elevated and there may be a need for preventative intervention in order to revert back to an “optimal” (i.e., green) state. Preventative interventions are designated for instances where a direct line of communication may need to be established with the worker to advise suggested actions, but there are no current emergency situations nor any need for coordination with EMS. Examples of lower risk events that could require a preventative intervention are: rising heat stress levels; small but rising remnants of flammable or volatile hazards detected by the atmospheric sensor; and minor physiological irregularities such as slightly higher/lower than usual heart rate or breathing rate. Going beyond mere preventative interventions, the detection of a more serious and/or time-sensitive situation is classified as an “emergency” (i.e., red) state, indicating that risk is beyond an acceptable threshold and immediate action should be taken (at a minimum, the worker should be removed from a confined space). Examples of higher risk and more time-sensitive events that require immediate intervention are: extreme heat stress levels during heat advisory weather conditions; rising levels of flammable or volatile hazards detected by the atmospheric sensor; and dangerous physiological symptoms such as excessively high/low heart rate while in a stationary position (e.g., 140 heartbeats per minute).

Based on health classifier assessments, the intervention response is then determined by the RSA, in many cases with the aid of CSS.

For many cases, particularly emergency situations, there may be a common and straightforward COA for an RSA to direct a worker to remove himself/herself from a confined space; or in more extreme cases, to call 911 to notify the EMS or fire department responsible for a given location. However, in more ambiguous and less time-sensitive circumstances, such as “sub-optimal” status indications described previously, the RSA may find it challenging to know all possible responses, particularly given the various possible health/safety hazards that could exist and their low frequency of occurrence. For this reason, a standardized ConOps for CSS may be formulated that employs a specific set of responses by system user depending on the color-coded alert. Specifically, there may be one COA defined per each alert level: blue, yellow, orange, and red. In virtually all cases, the Remote Attendant's (RA's) role is essential as the “eyes and ears” of the RSA on the production floor, and therefore is intended to be the first person to initially investigate the source of an alert, regardless of priority level. The CSS ConOps intends the RSA and RA to maintain continuous verbal communication via hand radios so that any alerts that occur are clearly and concisely communicated before taking the remedial action. Below are examples of high-level descriptions for each response according to the alert level.

Blue alert: Indicates that non-urgent help is requested by worker (typically manually initiated by worker), and that the issue does not implicate the worker's health and/or safety in any way. The RSA and RA provide a response as able, provided there are no higher severity alerts.

Yellow alert: Indicates the health status classifier detects a sub-optimal state for a specific worker but has not yet reached emergency levels that would merit contacting EMS. The purpose of this alert is to serve as an early warning. The RSA's and RA's goals are to provide immediate response in case the issue is trending toward a red state. If another red alert exists at this time, worker in yellow state must immediately evacuate the confined space.

Orange alert: Indicates the worker has a disconnection with their associated sensor kit that is blocking the desired level of remote health/safety monitoring. This issue may occur when the sensor battery level is low, the sensor is out of range, the mobile device powers off, or the BAS application crashes. The RSA's and RA's goals are to provide immediate response in case the issue is caused by an unsafe event and/or in case an incident occurs that cannot be detected due to the disconnection. If another yellow or red alert exists at this time, worker in orange state must evacuate the confined space; otherwise, the RA's goal is to assist with resolving the issue.

Red alert: Indicates the health status classifier detects an emergency level state for a specific worker. The RSA and RA must respond with full haste to confirm that an emergency has indeed occurred. The RA's goal is to reach the confined space entry hatch within 60 seconds and verify the issue. If a false alarm occurs, the RA verbally communicates this to RSA so the alert is promptly resolved and marked accordingly from the decision support station. Additionally, an optional feature to minimize response time is that EMS personnel (e.g., Fire Dept) are automatically notified of the red alert (if provided a CSS Geoview page at the EMS terminal station), so the exact location of the affected individual is provided. If the EMS does not receive a false alarm response within a certain amount of time (e.g., 90 seconds), they may automatically deploy to the scene. This ConOps decision shall be made by the applicable government Safety personnel.

Additionally, the RSA's decision support station includes a special system feature in which the RSA can initiate an “evacuate all” command to any workers working in a specific zone. In practice this is most similar to a red alert in that the primary goal is to immediately cease work activities and vacate the confined space. However, the evacuation command is unique in that it is initiated by the RSA, rather than being automated, and is intended to affect everyone working in confined spaces at a given time. The reason to issue evacuation commands can range from the presence of a volatile hazard that has entered a particular building (and thus affecting multiple confined spaces in that building) to the EMS or Fire Department teams being occupied and unavailable at a particular time.

To execute this ConOps and ensure the effective delivery of preventative health and safety interventions, it was also helpful to consider the most effective delivery medium to communicate a COA directly to both the RAs and the affected worker. In current depot operations, verbal radio communication is typically the single exclusive communication medium relied upon to provide directions. Although this has proven to be effective, it can often be slow and inefficient from the perspective of optimizing time, manpower, and attention, particularly for a single RSA responsible for monitoring dozens of other workers. For this reason, a part of CSS technical approach was to incorporate non-verbal methods of communication and information exchange through human-wearable technologies.

Wrist-worn devices, such as “smart watches,” for non-verbal communication fit well into the CSS concept. Furthermore, the ability to converge multiple CSS functions (i.e., motion sensors and worker UI) into a single non-invasive wearable device greatly improves user reception and overall effectiveness of the system. Although a smartwatch does not replace or adequately substitute verbal radio communication for many situations, under the right circumstances—such as requests to enter a confined space, requests for help, mass notifications (e.g., evacuate confined spaces), and basic acknowledgements—a non-verbal indicator sent via wrist-worn system interactions provides a faster and less costly means to track worker status information and convey information without affecting worker health/safety. The smartwatch may be holstered in an arm band form factor to avoid having to wear on a wrist.

The Samsung Galaxy Watch is an example means to unobtrusively measure continuous motion level on a worker through a work shift. The Galaxy Watch can run software that performs many of these functions, including: communicating your confined space entry status (e.g., pending approval to enter, approved to enter, inside space); calling for a service request (blue alert); calling for critical help (red alert); and receiving notifications when an alert has occurred. This Galaxy Watch application also provides a way to display a special form of alert—deemed an Aqua alert—that communicates directly to the worker that a pending alert is getting ready to trigger. The Aqua alert occurs prior to reaching the RSA decision support station so benign situations (e.g., no-motion alerts caused by sitting still for too long) are identified before causing a false alarm situation.

Similar benefits to those provided for worker communication can be realized by providing the smartwatch application to RAs. Specifically, when a worker in the RA's zone triggers an alert or call for help, the RA may receive this alert on their smartwatch UI. RSA's Geoview may be configured specifically for RA usage on a mobile device. This UI displays the RA's current location in the center of the screen and upon selecting the worker with an alert status, a line is overlaid to connect the RA's location to that worker. The distance (number of feet) to reach the affected worker may also be displayed.

To ensure RAs are not asked to cover an unreasonably sized area—an essential requirement to assure they can respond to alerts in the minimum amount of time needed—the CSS ConOps may force RAs to be assigned to a specific zone. If multiple zones are defined, then multiple RAs are needed to cover each zone at a one-to-one ratio. To support this requirement, all CSS Geoview displays may provide the option to display zone overlays. Zone configurations may be configurable by an administrator panel.

For emergency situations that require a time-sensitive response by an outside agency (e.g., local EMS or fire department), CSS may provide a data publishing service that supplies critical information (e.g., alert states, worker locations, number of workers in confined spaces) directly to the first response team. Although distribution of this information to response teams is an optional feature, it can reduce coordination time between the RSA, RA, maintenance control, and emergency responders. The data publishing service is facilitated by the use of the CSS cloud-based services and the use of secure web technologies that can be easily accessed through conventional web browsers (e.g., Google Chrome). Each applicable organization has the option of using one of the existing read-only versions of the CSS displays, such as the Geoview.

### Research Results from One Example Embodiment

CSS is a sensors-based system for remote health and safety monitoring of workers working in confined spaces. CSS may provide two aspects: an unobtrusive sensor suite worn by workers and an integrated decision support station for alerting and intervention. In the embodiment tested, the sensor suite collects real-time measurements of worker' health signals (heart rate, breathing, motion), atmospheric levels in their vicinity (oxygen, LEL, VOCs), and location in GPS-denied environments. A decision support station provides remote monitoring for a single safety attendant to safely monitor the health and safety of many confined spaces concurrently. This is designed especially for early-warning detection for preventative intervention and accelerating response by EMS personnel.

Embodiments of CSS utilize four classes of technical components: portable sensors, data networking, remote monitoring displays, and alerting and intervention. In the embodiment tested, the CSS packages portable sensors into “sensor kits” assigned to a specific worker prior to entering a confined space. A sensor kit may comprise health, atmospheric, and location sensors. The health sensors used with the current prototype are the Polar Team Pro base layer shirt, Polar H10 wireless unit, and Samsung Galaxy smartwatch with arm band holster. The Polar Team Pro and H10 unit collect real-time heart rate and R-R intervals from its user. The Samsung Galaxy smartwatch is used to measure real-time actigraphy (motion levels) from its user, while offering a communication display to receive alerts/notifications and calls for help as needed. The atmospheric sensor used with the current CSS prototype is RAE Systems' MultiRAE Pro, which measures atmospheric data from the worker's immediate surroundings. Location sensors are provided by TRX Systems' NEON indoor tracking system, a hybrid solution that uses BLE-based iBeacons, Ultra-Wideband beacons, MEMS-based inertial navigation, and additional constraints and pre-mappings to optimize accuracy.

The Gen1 data network comprises BLE short-range data communication, 4G LTE frequencies for long-range communication, and Amazon Web Services GovCloud for real-time processing and communication to the monitoring displays. BLE is used to connect each portable sensor to a mobile device running the BAS software program. The BAS mobile device manages the receipt, local processing, and relay of sensor data to the cloud server via a wireless network provider's 4G LTE (e.g., Verizon, AT&T). The cloud services manage real-time data processing, alert generation, data storage, and remote display access via a web server to approved clients on additional networks, such as Verizon MiFi Hotspots or AFNET.

This CSS embodiment provides three primary remote safety monitoring displays: Geoview, Primary Overview, and Worker Status. Because these are web-based client applications, they are accessible from a wide range of devices, including desktop computers, tablets, and cell phones. The Geoview overlays workers' location data on a map display along with their current health status, allowing a prompt and accurate response to their location if a potential emergency occurs. The Personnel Overview display provides a card-based view of each worker checked into CSS at a given time, while illustrating their current health status and detailed information on any system-generated alerts. The Personnel Overview display is also the RA's primary tool for navigating the overall remote monitoring station, offering features such as acknowledgement of confined space entries, worker selection, zone evacuation commands, alert management, and auto-sorting workers inside vs. outside confined spaces. The Status display allows viewing of a specific worker's sensor data at a greater level of detail than the previous displays. In particular, this display allows viewing of both current sensor readings and recent historical sensor data that may be relevant to the current health and safety status.

The alerting and intervention components of CSS comprise a backend alert generation module that drives specific display behavior on the Geoview, Personnel Overview, and Status displays. In response, the associated ConOps dictates the system end users to follow a specific intervention protocol to rapidly confirm (or deny, in false alarm instances) the existence of the detected event, followed by completion of all necessary steps to ensure the safety of the affected individual. The alert generation module follows a color-coded scheme tied to a specific intervention response by safety attendants. This current CSS embodiment is programmed to classify each worker as a Green state unless specific alerting criteria are met. The current embodiment contains a library of algorithms that each probe for a specific state, and if detected, the worker is classified into the new state accordingly. The algorithms library can be updated and expanded as further system testing is conducted and/or if new additional sensors are added to the worker sensor kits.

Although this invention has been described in the above forms with a certain degree of particularity, it is understood that the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention which is defined in the claims and their equivalents.

We claim:

1. A sensor system comprising:
a mounting base;
a body area subsystem comprising a sensor module;
the mounting base configured to removably couple the sensor module to the mounting base; and
the sensor module comprising:
a sensor subsystem,
a communication subsystem configured to transmit raw data from the sensor subsystem and receive contextualized data, and
a power subsystem configured to power the sensor subsystem and the communication subsystem.

2. The sensor system of claim 1 wherein:
the sensor module housing the power subsystem, the sensor subsystem and the communication subsystem; and
the sensor module is configured to removably couple the sensor module to the mounting base whereby a first sensor module with a first sensor coupled to the mounting base can be exchanged with a second sensor module with a second sensor.

3. The sensor system of claim 1 wherein:
the sensor module housing the power subsystem, the sensor subsystem and the communication subsystem;
the sensor module is configured to removably couple the sensor module to the mounting base whereby a first sensor module with a first sensor coupled to the mounting base can be exchanged with a second sensor module with a second sensor; and
the mounting base is configured to be mounted on a limb of a worker whereby the first sensor module can be exchanged with the second sensor module while the mounting base is mounted on the limb of the worker.

4. The sensor system of claim 1 wherein the sensor subsystem comprises an optical sensor.

5. The sensor system of claim 1 wherein:
the sensor subsystem comprises an optical sensor; and
the mounting base comprising an opening configured to allow the optical sensor to be proximal to a portion of a skin of a wearer when the optical sensor is coupled to the mounting base and the mounting base is mounted on a limb of a wearer.

6. The sensor system of claim 1 wherein the sensor subsystem comprises a sensor selected from the group consisting of:
an environmental sensor;
a location sensor;
a physiological sensor;
a behavior sensor; and
a posture sensor.

7. The sensor system of claim 2 wherein the mounting base further comprises a base coupling element configured to removably couple the sensor module to the mounting base.

8. The sensor system of claim 7 wherein:
the sensor module further comprises a sensor module coupling element configured to removably couple the sensor module to a component block;
the component block comprising a component block coupling element configured to removably mate with the sensor module coupling element;
the component block coupling element comprises one or more pliant clasp; and
the sensor module coupling element comprises one or more recesses to receive a portion of the pliant clasp.

9. The sensor system of claim 8 wherein the component block comprises one selected from the group consisting of:
a sensor component block;
a communication component block;
a power component block; and
a battery clock component block.

10. The sensor system of claim 8 wherein the component block comprises a sensor component block comprising a sensor selected from the group consisting of:
an environmental sensor;
a location sensor;
a physiological sensor;
a behavior sensor; and
a posture sensor.

11. The sensor system of claim 8 wherein the component block comprises a communication component block comprising a communication protocol module programmed to communicate according to a communication protocol selected from the group consisting of:
a Bluetooth protocol;
a 802.11 protocol; and
a LoRa protocol.

12. The sensor system of claim 1 further comprising a monitoring subsystem configured to determine a contextualized state of a worker from a sensor data from the sensor module.

13. The sensor system of claim 1 further comprising a monitoring subsystem comprising a situation classifier configured to determine a contextualized state of a worker from a sensor data from the sensor module.

14. The sensor system of claim 1 further comprising:
a monitoring subsystem in communication with the communication subsystem;
the monitoring subsystem configured to receive a first sensor data from a first sensor of the sensor module for a time and a second sensor data from a second sensor of the sensor module for the time;
the monitoring subsystem comprising:
an expert subsystem comprising a situation classifier module and an intervention/alert module,
a subsystem database comprising contextualization rules and a library of alert rules,
the situation classifier module is configured to determine a first contextualized situation at the time from the first sensor data using the contextualization rules, the situation classifier module is configured to determine a second contextualized situation at the time from the first sensor data and the second sensor data using the contextualization rules,
the intervention/alert module configured to compare the first contextualized situation to the library of alert rules to determine whether an alert situation has occurred at the time, and
the intervention/alert module configured to compare the second contextualized situation to the library of alert rules to determine whether the alert situation has occurred at the time;
whereby if the alert situation has occurred at the time based on the first contextualized situation, but the alert situation has not occurred at the time based on the second contextualized situation, the monitoring subsystem configured to not communicate an alert; and
whereby if the alert situation has occurred at the time based on the first contextualized situation, and the alert situation has occurred at the time based on the second contextualized situation, the monitoring subsystem configured to communicate an alert.

15. A processor based contextualized sensor system comprising:
a body area subsystem comprising:
a first sensor configured to communicate a first sensor data from a time, and
a second sensor configured to communicate a second sensor data from the time; a monitoring subsystem comprising:
a message broker configured to receive the first sensor data, the second sensor data and the time from the body area subsystem,
an expert subsystem comprising a situation classifier module and an intervention/alert module,
a subsystem database comprising contextualization rules and a library of alert rules,
the situation classifier module is configured to determine a first contextualized situation at the time from the first sensor data using the contextualization rules, the situation classifier module is configured to determine a second contextualized situation at the time from the first sensor data and the second sensor data using the contextualization rules,
the intervention/alert module configured to compare the first contextualized situation to the library of alert rules to determine whether an alert situation has occurred at the time, and
the intervention/alert module configured to compare the second contextualized situation to the library of alert rules to determine whether the alert situation has occurred at the time;
whereby if the alert situation has occurred at the time based on the first contextualized situation, but the alert situation has not occurred at the time based on the second contextualized situation, the monitoring subsystem configured to not communicate an alert; and
whereby if the alert situation has occurred at the time based on the first contextualized situation, and the alert situation has occurred at the time based on the second contextualized situation, the monitoring subsystem configured to communicate an alert.

16. The processor based contextualized sensor system of claim 15 wherein:
the first sensor comprises a physiological sensor configured to communicate a physiological data as the first sensor data;
the situation classifier module further comprises a state contextualizer module configured to determine a first contextualized state of a worker at the time from the first sensor data and a second contextualized state of the worker at the time from the first sensor data and the second sensor data;
the first contextualized situation comprises the first contextualized state of the worker at the time; and
the second contextualized situation comprises the second contextualized state of the worker at the time.

17. The processor based contextualized sensor system of claim 15 wherein:
the body area subsystem further comprises a mounting base; and
the first sensor and the second sensor are configured to be removably coupled to the mounting base.

18. A processor based contextualized sensor system for determining a contextualized state of a worker, the system comprising:
a body area subsystem comprising:
a mounting base,
a sensor module comprising one or more sensors configured to collect a state data of a worker,
the state data comprising a first raw state data at a time and a second raw state data at the time, and
a communication subsystem configured to communicate the first raw state data,
the second raw state data and the time;
a monitoring subsystem comprising:
a message broker configured to receive the first raw state data, the second raw state data and the time from the body area subsystem,
an expert subsystem comprising a situation classifier module,
a subsystem database comprising contextualization rules, and
the situation classifier module is configured to determine a raw state of the worker at the time from the first raw state data and determine a contextualized state of the worker at the time from the first raw state data at the time and the second raw state data at the time and the contextualization rules;
the contextualization rules comprise a raw variable and a contextualization variable to identify a resulting contextualized variable;
the raw variable comprises the first raw state data of the worker at the time;
the contextualization variable comprises the second raw state data of the worker at the time;
the resulting contextualized variable comprises an objective representation of the contextualized state of the worker at the time given the first raw state data at the time and the second raw state data of the worker at the time; and
the situation classifier module is configured to use the contextualization rules to (1) confirm the raw state of the worker at the time as the contextualized state of the worker at the time or (2) override the raw state of the worker at the time as the contextualized state of the worker at the time.

19. The processor based contextualized sensor system of claim 18 wherein:
the sensor module comprises a first sensor and a second sensor; and
the first sensor and the second sensor are configured to be removably coupled to the mounting base.
